(19) United States
(12) Patent Application Publication
(10) Pub. No.: US 2024/0091364 A1
(43) Pub. Date: Mar. 21, 2024

(54) CANCER COMBINATION TREATMENTS USING ANTI-STAT3 NUCLEIC ACID CONJUGATES

(71) Applicant: CITY OF HOPE, Duarte, CA (US)

(72) Inventors: Hua YU, Glendora, CA (US); Chunyan ZHANG, Glendora, CA (US)

(21) Appl. No.: 17/767,398

(22) PCT Filed: Oct. 9, 2020
(86) PCT No.: PCT/US20/55102

§ 371 Date: Apr. 7, 2022

Related U.S. Application Data
(60) Provisional application No. 62/913,102, filed on Oct. 9, 2019.

(51) Int. Cl. A61K 47/54; A61K 39/395; A61P 35/00

(52) U.S. Cl. CPC A61K 47/549; A61K 39/3955; A61P 35/00; A61K 2039/505

(57) ABSTRACT

Provided herein are, inter ilia, methods and compositions for the treatment of cancer. The methods include administering a combination of an anti-STAT3-Toll-like receptor 9 (TLR9)-binding conjugate and a checkpoint inhibitor. The anti-STAT3-Toll-like receptor 9 (TLR9)-binding conjugate provide herein may be a CpG moiety bound to an anti-STAT3 siRNA through a covalent linker and the checkpoint inhibitor may be an anti-CTLA4 antibody. Administration of a combined effective amount of the anti-STAT3-Toll-like receptor 9 (TLR9)-binding conjugate and the checkpoint inhibitor results in surprisingly increased anti-tumor efficacy and CDS T cell activity.

### CROSS-REFERENCES TO RELATED APPLICATIONS

This application claims the benefit U.S. Provisional Application Ser. No. 62/913,102, filed Oct. 9, 2019, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant no. P50 CA107399 awarded by the City of Hope and NIH/NCI. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048440-734001WO_SEQUENCE_LISTING_ST25.TXT, created on Sep. 25, 2020, 7,348 bytes, machine format IBM-PC, MS-Windows operating system, is hereby incorporated by reference.

### BACKGROUND

Recent promising human results of immunotherapies to block immune checkpoints such as cytotoxic T-lymphocyte-associated antigen 4 (CTLA4) and programmed cell death protein 1 (PD-1) (Pardoll, D. M., Nat Rev Cancer 12:252-264 (2012); Pardoll, D. M., Nat Immunol 13:1129-1132 (2012); Keir, M. E. et al., Annu Rev Immunol 26:677-704 (2008)) illustrate the importance of targeting molecules that inhibit T cell-mediated antitumor immunity. However, the immunosuppressive tumor microenvironment hampers the success of various immunotherapies. There are several intracellular checkpoints with great potential as targets to promote potent antitumor immunity. STAT3, for example, has been shown to be a crucial signaling mediator in tumor-associated immune cells, as well as in tumor cells (Yu, H. et al., Nat Rev Cancer 9:798-809 (2009); Kortylewski, M. and Yu, H., Curr Opin Immunol 20:228-233 (2008); Kortylewski, M. et al., Nat Med 11:1314-1321 (2005); Herrmann, A. et al., Cancer Res 70:7455-7464 (2010)). In tumor cells, STAT3 promotes tumor cell survival/proliferation, invasion, and immunosuppression (Yu, H., and Jove, R., Nat Rev Cancer 4:97-105 (2004)). In the tumor microenvironment, STAT3 is persistently activated in immune cells, including T cells (Kujawski, M. et al., Cancer Res 70:9599-9610 (2010); Yu, H. et al., Nat Rev Immunol 7:41-51 (2007)). CD4⁺ T regulatory cells (T_{Regs}) can induce peripheral tolerance, suppressing CD8 T cell functions in various diseases including cancer (Kortylewski, M. et al., Nat Med 11:1314-1321 (2005); Curiel, T. J. et al., Nat Med 10:942-949 (2004); Shevach, E. M., Nat Rev Immunol 2:389-400 (2002); Chen, M. L. et al., Proc Natl Acad Sci USA 102:419-424 (2005); Mempel, T. R. et al., Immunity 25:129-141 (2006); Arens, R. and Schoenberger, S. P., Immunol Rev 235:190-205 (2010)). Activated STAT3 in T cells contributes to expanding tumor-associated CD4⁺ T_{Regs} (Kortylewski, M. et al., Nat Med 11:1314-1321 (2005); Pallandre, J. R. et al., J Immunol 179:7593-7604 (2007)). Moreover, Stat3^{-/-} CD8⁺ T cells, both endogenous or adoptively transferred, mount potent antitumor immune responses compared to those with intact Stat3 (Kujawski, M. et al., Cancer Res 70:9599-9610 (2010)).

As a nuclear transcription factor lacking its own enzymatic activity, STAT3 is a challenging target for both antibody and small-molecule drugs (Yu, H., and Jove, R., Nat Rev Cancer 4:97-105 (2004); Darnell, J. E., Nat Med 11:595-596 (2005); Darnell, J. E., Jr., Nat Rev Cancer 2:740-749 (2002)). Recent pioneering work has shown the feasibility to deliver siRNA into tumor cells in vivo (McNamara, J. O., 2nd et al., Nat Biotechnol 24:1005-1015 (2006)). In particular, chimeric RNAs or DNA-RNAs consisting of a siRNA fused to nucleic acid sequences, which bind to either a cell surface ligand or an intracellular receptor with high affinity, have demonstrated therapeutic efficacy in preclinical models (McNamara, J. O., 2nd et al., Nat Biotechnol 24:1005-1015 (2006); Wheeler, L. A. et al., J Clin Invest 121:2401-2412 (2011); Kortylewski, M. et al., Nat Biotechnol 27:925-932 (2009)). The majority of such siRNA delivery technologies involves the fusion of siRNa to an aptamer, a structured RNA with high affinity to epitopes on tumor cells and virally infected epithelial cells. Applicants recently described a technology for efficient in vivo delivery of siRNA into immune cells by linking an siRNA to CpG oligonucleotide, which binds to its cognate receptor, TLR9 (Kortylewski, M. et al., Nat Biotechnol 27:925-932 (2009)). TLR9 is expressed intracellularly in cells of myeloid lineage and B cells, as well as tumor cells expressing TLR9, including human leukemic cells (Kortylewski, M. et al., Nat Biotechnol 27:925-932 (2009); Zhang, Q. et al., Blood 121:1304-1315 (2013)). However, the CpG-siRNA approach does not directly target T cells (Kortylewski, M. et al., Nat Biotechnol 27:925-932 (2009)). The methods and compositions provided herein cure these and other needs in the art.

### BRIEF SUMMARY OF THE INVENTION

In an aspect, a method of treating a cancer in a subject in need thereof is provided. The method includes administering to the subject an effective amount of an anti-STATS-Toll-like receptor 9 (TLR9)-binding conjugate and an effective amount of a checkpoint inhibitor.

In an aspect, a method of treating a cancer in a subject in need thereof is provided. The method includes administering to the subject an effective amount of an anti-STATS-Toll-like receptor 9 (TLR9)-binding conjugate including a CpG moiety bound to an anti-STAT3 siRNA through a covalent linker and an effective amount of an anti-CTLA4 antibody.

In an aspect, a method of treating a cancer in a subject in need thereof is provided. The method includes administering to the subject an anti-STAT3-Toll-like receptor 9 (TLR9)-binding conjugate and a checkpoint inhibitor in a combined effective amount.

In an aspect a method of treating a cancer in a subject in need thereof is provided. The method includes administering to the subject: (i) an anti-STAT3-TLR9 binding conjugate including a CpG moiety bound to an anti-STAT3 siRNA through a covalent linker; and (ii) an anti-CTLA4 antibody, in a combined effective amount.

In an aspect, a method of treating a cancer in a subject in need thereof is provided. The method includes administering to the subject an effective amount of an anti-STAT3-Toll-like receptor 9 (TLR9)-binding conjugate and an effective amount of a PARP inhibitor.

In an aspect, a method of treating a cancer in a subject in need thereof is provided. The method includes administering to the subject an anti-STAT3-Toll-like receptor 9 (TLR9)-binding conjugate and a PARP inhibitor in a combined effective amount.

In another aspect, a pharmaceutical composition is provided. The composition includes an anti-STAT3-Toll-like receptor 9 (TLR9)-binding conjugate, a checkpoint inhibitor and a pharmaceutically acceptable excipient, wherein the anti-STAT3-TLR9-binding conjugate and the checkpoint inhibitor are present in a combined synergistic amount, wherein the combined synergistic amount is effective to treat cancer in a subject in need thereof.

In another aspect, a pharmaceutical composition is provided. The composition includes an anti-STAT3-Toll-like receptor 9 (TLR9)-binding conjugate, a checkpoint inhibitor and a pharmaceutically acceptable excipient, wherein the anti-STAT3-TLR9-binding conjugate and the checkpoint inhibitor are present in a combined effective amount, wherein the combined effective amount is effective to treat cancer in a subject in need thereof.

In another aspect, a pharmaceutical composition is provided. The composition includes an anti-STAT3-Toll-like receptor 9 (TLR9)-binding conjugate, a PARP inhibitor and a pharmaceutically acceptable excipient, wherein the anti-STAT3-TLR9-binding conjugate and the PARP inhibitor are present in a combined synergistic amount, wherein the combined synergistic amount is effective to treat cancer in a subject in need thereof.

In another aspect, a pharmaceutical composition is provided. The composition includes an anti-STAT3-Toll-like receptor 9 (TLR9)-binding conjugate, a PARP inhibitor and a pharmaceutically acceptable excipient, wherein the anti-STAT3-TLR9-binding conjugate and the PARP inhibitor are present in a combined effective amount, wherein the combined effective amount is effective to treat cancer in a subject in need thereof.

In another aspect, a pharmaceutical composition is provided. The pharmaceutical composition includes an anti-STAT3-TLR9 binding conjugate including a CpG moiety bound to an anti-STAT3 siRNA through a covalent linker, an anti-CTLA4 antibody and a pharmaceutically acceptable excipient, wherein the anti-STAT3-TLR9-binding conjugate and the anti-CTLA4 antibody are present in a combined synergistic amount, wherein the combined synergistic amount is effective to treat cancer in a subject in need thereof.

In another aspect, a pharmaceutical composition is provided. The pharmaceutical composition includes an anti-STAT3-TLR9 binding conjugate including a CpG moiety bound to an anti-STAT3 siRNA through a covalent linker, an anti-CTLA4 antibody and a pharmaceutically acceptable excipient, wherein the anti-STAT3-TLR9-binding conjugate and the anti-CTLA4 antibody are present in a combined effective amount, wherein the combined effective amount is effective to treat cancer in a subject in need thereof.

### BRIEF DESCRIPTION OF THE DRAWINGS

FIGS. 1A-1C present bar graphs depicting combination treatment with CpG-STAT3 siRNA and CTLA4 blockade that potently and significantly enhances the antitumor effector functions of tumor infiltrating T cells. Single cell suspensions prepared from tumors in A20 tumor bearing mice were analyzed by flow cytometry for IFNγ (FIGS. 1A-1B) and granzyme B expression (FIG. 1C) in T cells. Statistical results of flow cytometry analysis showing IFNγ⁺ or GzmB⁺ cells frequencies in CD4⁺ or CD8⁺ T cells. In this figure, significance is shown as follows: * p<0.05, ** p<0.01, and *** p<0.001.

FIG. 2 is a line graph depicting that the therapeutic effect of CpG-STAT3 siRNA can be augmented by combination with CTLA4 blockade. BALB/c mice with subcutaneous (s.c) A20 lymphoma were treated by peritumoral injections of CpG-STAT3 siRNA, i.p. injection of αCTLA4 every other day, starting 10 days after challenge with 5×10⁵ A20 cells, n=5. Tumor size was monitored for every other day. SE and significance shown?

FIG. 3 is a line graph depicting the therapeutic effect of CpG-STAT3 siRNA treatment on human T cell lymphoma. NSG mice with subcutaneous (s.c) H9 human T cell lymphoma were treated by intratumoral injections of PBS, CpG-ODND¹⁹ or CpG(D19)-STAT3 siRNA, every other day, starting 10 day after challenge with 5×106 H9 cells, n=6-7 mice. Tumor size was monitored for every other day. SE and significance shown (*** P<0.001).

### DETAILED DESCRIPTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from an heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzooxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocycloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,”, “cycloalkyl”, “heterocycloalkyl”, “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂N(R)(′R″—NRSO₂R′), —CN, and —NO₂ in a number ranging from zero to (2m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, NR″C(O)₂R′, NRC(NR′R″)═NR′″, S(O)R′, —S(O)₂R′, —S(O)₂N(R′)(R″, —NRSO₂R′), —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Where a moiety is substituted with an R substituent, the group may be referred to as “R-substituted.” Where a moiety is R-substituted, the moiety is substituted with at least one R substituent and each R substituent is optionally different. For example, where a moiety herein is R^{1A}-substituted or unsubstituted alkyl, a plurality of R^{1A} substituents may be attached to the alkyl moiety wherein each R^{1A} substituent is optionally different. Where an R-substituted moiety is substituted with a plurality of R substituents, each of the R-substituents may be differentiated herein using a prime symbol (′) such as R′, R″, etc. For example, where a moiety is R^{3A}-substituted or unsubstituted alkyl, and the moiety is substituted with a plurality of R^{3A} substituents, the plurality of R^{3A} substituents may be differentiated as R^{3A}′, R^{3A}″, R^{3A}′″, etc. In some embodiments, the plurality of R substituents is 3.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where variables s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
   (i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, heteroaryl, substituted with at least one substituent selected from:
      (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

As used herein, the term “conjugate” refers to the association between atoms or molecules. The association can be direct or indirect. For example, a conjugate between a nucleic acid and a protein can be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, conjugates are formed using conjugate chemistry including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful reactive moieties or functional groups used for conjugate chemistries (including “click chemistries” as known in the art) herein include, for example:
(a) carboxyl groups and various derivatives thereof including, but not limited to, N-hydroxysuccinimide esters, N-hydroxybenzotriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold;
(h) amine or sulfhydryl groups, which can be, for example, acylated, alkylated or oxidized;
(i) alkenes, which can undergo, for example, cycloadditions, acylation, Michael addition, etc.;
(j) epoxides, which can react with, for example, amines and hydroxyl compounds;
(k) phosphoramidites and other standard functional groups useful in nucleic acid synthesis;
(l) metal silicon oxide bonding;
(m) metal bonding to reactive phosphorus groups (e.g. phosphines) to form, for example, phosphate diester bonds; and
(n) sulfones, for example, vinyl sulfone.

Chemical synthesis of compositions by joining small modular units using conjugate (“click”) chemistry is well known in the art and described, for example, in H. C. Kolb, M. G. Finn and K. B. Sharpless ((2001). “Click Chemistry: Diverse Chemical Function from a Few Good Reactions”. Angewandte Chemie International Edition 40 (11): 2004-2021); R. A. Evans ((2007). “The Rise of Azide-Alkyne 1,3-Dipolar ‘Click’ Cycloaddition and its Application to Polymer Science and Surface Modification”. Australian Journal of Chemistry 60 (6): 384-395; W. C. Guida et al. Med. Res. Rev. p 3 1996; Spiteri, Christian and Moses, John E. ((2010). “Copper-Catalyzed Azide-Alkyne Cycloaddition: Regioselective Synthesis of 1,4,5-Tri substituted 1,2,3-Triazoles”. Angewandte Chemie International Edition 49 (1): 31-33); Hoyle, Charles E. and Bowman, Christopher N. ((2010). “Thiol-Ene Click Chemistry”. Angewandte Chemie International Edition 49 (9): 1540-1573); Blackman, Melissa L. and Royzen, Maksim and Fox, Joseph M. ((2008). “Tetrazine Ligation: Fast Bioconjugation Based on Inverse-Electron-Demand Diels-Alder Reactivity”. Journal of the American Chemical Society 130 (41): 13518-13519); Devaraj, Neal K. and Weissleder, Ralph and Hilderbrand, Scott A. ((2008). “Tetrazine Based Cycloadditions: Application to Pretargeted Live Cell Labeling”. Bioconjugate Chemistry 19 (12): 2297-2299); Stockmann, Henning; Neves, Andre; Stairs, Shaun; Brindle, Kevin; Leeper, Finian ((2011). “Exploring isonitrile-based click chemistry for ligation with biomolecules”. Organic & Biomolecular Chemistry), all of which are hereby incorporated by reference in their entirety and for all purposes.

The reactive functional groups can be chosen such that they do not participate in, or interfere with, the chemical stability of the proteins or nucleic acids described herein. By way of example, the nucleic acids can include a vinyl sulfone or other reactive moiety (e.g., maleimide). Optionally, the nucleic acids can include a reactive moiety having the formula —S—S—R. R can be, for example, a protecting group. Optionally, R is hexanol. As used herein, the term hexanol includes compounds with the formula C₆H₁₃OH and includes, 1-hexanol, 2-hexanol, 3-hexanol, 2-methyl-1-pentanol, 3-methyl-1-pentanol, 4-methyl-1-pentanol, 2-methyl-2-pentanol, 3-methyl-2-pentanol, 4-methyl-2-pentanol, 2-methyl-3-pentanol, 3-methyl-3-pentanol, 2,2-dimethyl-1-butanol, 2,3-dimethyl-1-butanol, 3,3-dimethyl-1-butanol, 2,3-dimethyl-2-butanol, 3,3-dimethyl-2-butanol, and 2-ethyl-1-butanol. Optionally, R is 1-hexanol.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

The terms “isolate” or “isolated”, when applied to a nucleic acid, virus, or protein, denotes that the nucleic acid, virus, or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. In embodiments, a nonspecific sequence is a nucleic acid sequence lacking transcriptional or translational function. By way of example, a nonspecific nucleic acid sequence is a sequence of nucleic acid residues that does not function as an inhibitory nucleic acid when contacted with a cell or organism.

An “antisense nucleic acid” as referred to herein is a nucleic acid (e.g., DNA or RNA molecule) that is complementary to at least a portion of a specific target nucleic acid and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid. In embodiments, the antisense nucleic acid hybridizes to the target nucleic acid in vitro. In embodiments, the antisense nucleic acid hybridizes to the target nucleic acid in a cell. In embodiments, the antisense nucleic acid hybridizes to the target nucleic acid in an organism. In embodiments, the antisense nucleic acid hybridizes to the target nucleic acid under physiological conditions. Antisense nucleic acids may comprise naturally occurring nucleotides or modified nucleotides such as, e.g., phosphorothioate, methylphosphonate, and -anomeric sugar-phosphate, backbone-modified nucleotides.

In the cell, the antisense nucleic acids hybridize to the corresponding RNA forming a double-stranded molecule. The antisense nucleic acids interfere with the endogenous behavior of the RNA and inhibit its function relative to the absence of the antisense nucleic acid. Furthermore, the double-stranded molecule may be degraded via the RNAi pathway. The use of antisense methods to inhibit the in vitro translation of genes is well known in the art (Marcus-Sakura, Anal. Biochem., 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), or certain of their derivatives or pre-cursors.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

As used herein, the term “conjugated” when referring to two moieties means the two moieties are bonded, wherein the bond or bonds connecting the two moieties may be covalent or non-covalent. In embodiments, the two moieties are covalently bonded to each other (e.g. directly or through a covalently bonded intermediary). In embodiments, the two moieties are non-covalently bonded (e.g. through ionic bond(s), van der Waal's bond(s)/interactions, hydrogen bond(s), polar bond(s), or combinations or mixtures thereof).

Nucleic acids may include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism. An “inhibitory nucleic acid” is a nucleic acid (e.g. DNA, RNA, polymer of nucleotide analogs) that is capable of binding to a target nucleic acid (e.g. an mRNA translatable into a protein) and reducing transcription of the target nucleic acid (e.g. mRNA from DNA) or reducing the translation of the target nucleic acid (e.g. mRNA) or altering transcript splicing (e.g. single stranded morpholino oligo).

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

A “labeled nucleic acid or oligonucleotide” is one that is bound, either covalently, through a linker or a chemical bond, or noncovalently, through ionic, van der Waals, electrostatic, or hydrogen bonds to a label such that the presence of the nucleic acid may be detected by detecting the presence of the detectable label bound to the nucleic acid. Alternatively, a method using high affinity interactions may achieve the same results where one of a pair of binding partners binds to the other, e.g., biotin, streptavidin. In embodiments, the phosphorothioate nucleic acid or phosphorothioate polymer backbone includes a detectable label, as disclosed herein and generally known in the art.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA or RNA sequences being linked are near each other, and, may be contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

A “siRNA,” “small interfering RNA,” “small RNA,” or “RNAi” as provided herein refers to a nucleic acid that forms a double stranded RNA, which double stranded RNA has the ability to reduce or inhibit expression of a gene or target gene when expressed in the same cell as the gene or target gene. The complementary portions of the nucleic acid that hybridize to form the double stranded molecule typically have substantial or complete identity. In one embodiment, a siRNA or RNAi refers to a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length. Non-limiting examples of siRNAs include ribozymes, RNA decoys, short hairpin RNAs (shRNA), micro RNAs (miRNA) and small nucleolar RNAs (snoRNA). In embodiments, the siRNA has a 2′chemical modification. In embodiments, the siRNA has serum stability-enhancing chemical modification, e.g., a phosphothioate internucleotide linkage, a 2′-O-methyl ribonucleotide, a 2′-deoxy-2′fluoro ribonucleotide, a 2′-deoxy ribonucleotide, a universal base nucleotide, a 5-C methyl nucleotide, an inverted deoxybasic residue incorporation, or a locked nucleic acid. In embodiments, the siRNA hybridizes to the corresponding mRNA sequence. Full complementarity is not necessarily required, provided there is sufficient complementarity to cause hybridization. In some embodiments, the degree of complementarity between a siRNA and its corresponding target RNA sequence, when optimally aligned using a suitable alignment algorithm, is about or more than about 50%, 60%, 75%, 80%, 85%, 90%, 91%, 92%, 93%, 94%, 95%, 96%, 97%, 98%, 99%, or 100%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif), SOAP (available at soap.genomics.org.cn), and Maq (available at maq.sourceforge.net). In embodiments, the siRNA has at least 80%, 85%, 90%, 95%, 96%, 97%, 98%, 99% or 100% sequence identity with the perfectly complementary sequence of the target RNA sequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “peptidyl” and “peptidyl moiety” means a monovalent peptide.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As to amino acid sequences, one of skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain. In embodiments, the amino acid side chain is H,

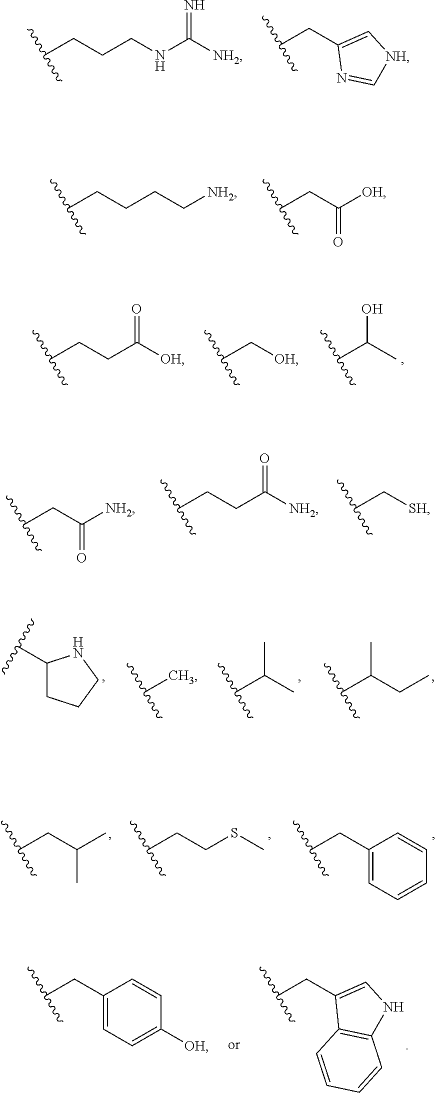

The term “non-natural amino acid side chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutyric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-Aminobicyclo[2.2.1]hept-5-ene-2-carboxylic acid hydrochloride, cis-2-Aminocycloheptanecarboxylic acid hydrochloride, cis-6-Amino-3-cyclohexene-1-carboxylic acid hydrochloride, cis-2-Amino-2-methylcyclohexanecarboxylic acid hydrochloride, cis-2-Amino-2-methylcyclopentanecarboxylic acid hydrochloride, 2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-Homopyr-OH, Boc-(2-indanyl)-Gly-OH, 4-Boc-3-morpholineacetic acid, 4-Boc-3-morpholineacetic acid, Boc-pentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH₂)—OH, Boc-Phe(3-NO2)-OH, Boc-Phe(3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]acetic acid purum, Boc-β-(2-quinolyl)-Ala-OH, N-Boc-1,2,3,6-tetrahydro-2-pyridinecarboxylic acid, Boc-β-(4-thiazolyl)-Ala-OH, Boc-β-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)—OH, Fmoc-Phe(4-Br)—OH, Fmoc-Phe(3,5-F2)-OH, Fmoc-β-(4-thiazolyl)-Ala-OH, Fmoc-β-(2-thienyl)-Ala-OH, 4-(Hydroxymethyl)-D-phenylalanine.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “small molecule” is used in accordance with its well understood meaning and refers to a low molecular weight organic compound that may regulate a biological process. In embodiments, the small molecule is a compound that weighs less than 900 daltons. In embodiments, the small molecule weighs less than 800 daltons. In embodiments, the small molecule weighs less than 700 daltons. In embodiments, the small molecule weighs less than 600 daltons. In embodiments, the small molecule weighs less than 500 daltons. In embodiments, the small molecule weighs less than 450 daltons. In embodiments, the small molecule weighs less than 400 daltons.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

For specific proteins described herein (e.g., STAT3, FoxP3, CTLA-4), the named protein includes any of the protein's naturally occurring forms, or variants that maintain the protein transcription factor activity (e.g., within at least 50%, 80%, 90%, 95%, 96%, 97%, 98%, 99% or 100% activity compared to the native protein). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference or functional fragment thereof.

A “STAT3 gene” as referred to herein includes any of the recombinant or naturally-occurring forms of the gene encoding Signal transducer and Activator of transcription 3 (STAT3) or variants thereof that maintain STAT3 expression (e.g. within at least 50%, 80%, 90%, 95%, 96%, 97%, 98%, 99% or 100% expression level compared to STAT3). In some aspects, variants have at least 90%, 95%, 96%, 97%, 98%, 99% or 100% nucleotide sequence identity across the whole sequence or a portion of the sequence (e.g. a 1,000, 2,500, 5,000, 7,500, 10,000, 20,000, 30,000, 40,000, 50,000, 60,000, 70,000, 80,000, 81,000, 81,500, 82,000, 82,500, 83,500, 84,500, or 85,000 continuous nucleotide portion) compared to a naturally occurring STAT3 gene. In embodiments, variants have about 90%, 95%, 96%, 97%, 98%, 99% or 100% nucleotide sequence identity across the whole sequence or a portion of the sequence (e.g. about a 500, 1,000, 2,500, 5,000, 7,500, 10,000, 20,000, 30,000, 40,000, 50,000, 60,000, 70,000, 80,000, 81,000, 81,500, 82,000, 82,500, 83,500, 84,500, or 85,000 continuous nucleotide portion) compared to a naturally occurring STAT3 gene. In embodiments, the STAT3 gene is substantially identical to the nucleic acid identified by the NCBI reference number Gene ID: 6774 or a variant having substantial identity thereto. In embodiments, the STAT3 gene is substantially identical to the nucleic acid identified by the NCBI reference number NG 007370.1 or a variant having substantial identity thereto.

The term “CTLA-4” or “CTLA-4 protein” as provided herein includes any of the recombinant or naturally-occurring forms of the cytotoxic T-lymphocyte-associated protein 4 (CTLA-4) or variants or homologs thereof that maintain CTLA-4 protein activity (e.g. within at least 50%, 80%, 90%, 95%, 96%, 97%, 98%, 99% or 100% activity compared to CTLA-4). In some aspects, the variants or homologs have at least 90%, 95%, 96%, 97%, 98%, 99% or 100% amino acid sequence identity across the whole sequence or a portion of the sequence (e.g. a 50, 100, 150, 200 or 205, 210, 215, 220, 225, 230, 235, 240, 245, or 250 continuous amino acid portion) compared to a naturally occurring CTLA-4 polypeptide. In embodiments, the variants or homologs have about 90%, 95%, 96%, 97%, 98%, 99% or 100% amino acid sequence identity across the whole sequence or a portion of the sequence (e.g. about 50, 100, 150, 200 or 205, 210, 215, 220, 225, 230, 235, 240, 245, or 250 continuous amino acid portion) compared to a naturally occurring CTLA-4 polypeptide. In embodiments, CTLA-4 is the protein as identified by the NCBI sequence reference GI:83700231, homolog or functional fragment thereof.

The term “PD-1” or “PD-1 protein,” also known as cluster of differentiation 279 (CD279) as provided herein includes any of the recombinant or naturally-occurring forms of the programmed cell death protein 1 (PD-1) or variants or homologs thereof that maintain PD-1 protein activity (e.g. within at least 50%, 80%, 90%, 95%, 96%, 97%, 98%, 99% or 100% activity compared to PD-1). In some aspects, the variants or homologs have at least 90%, 95%, 96%, 97%, 98%, 99% or 100% amino acid sequence identity across the whole sequence or a portion of the sequence (e.g. a 50, 100, 150, 200, 250, 260, 265, 270, 275, 280, 285, 290, 295, 300, 305, 310, 315, 320, 325, 330, 335 or 340 continuous amino acid portion) compared to a naturally occurring PD-1 polypeptide. In embodiments, the variants or homologs have about 90%, 95%, 96%, 97%, 98%, 99% or 100% amino acid sequence identity across the whole sequence or a portion of the sequence (e.g. about 50, 100, 150, 200, 250, 260, 265, 270, 275, 280, 285, 290, 295, 300, 305, 310, 315, 320, 325, 330, 335 or 340 continuous amino acid portion) compared to a naturally occurring PD-1 polypeptide. In embodiments, PD-1 is the protein as identified by the NCBI sequence reference GI: 167857792, homolog or functional fragment thereof. In embodiments, PD-1 is the protein as identified by the NCBI sequence reference GI: 765526773, homolog or functional fragment thereof. In embodiments, PD-1 is the protein as identified by the NCBI sequence reference GI: 765526771, homolog or functional fragment thereof.

The term “PD-L1” or “PD-L1 protein,” also known as cluster of differentiation 274 (CD274) as provided herein includes any of the recombinant or naturally-occurring forms of the programmed death-ligand 1 (PD-L1) or variants or homologs thereof that maintain PD-L1 protein activity (e.g. within at least 50%, 80%, 90%, 95%, 96%, 97%, 98%, 99% or 100% activity compared to PD-L1). In some aspects, the variants or homologs have at least 90%, 95%, 96%, 97%, 98%, 99% or 100% amino acid sequence identity across the whole sequence or a portion of the sequence (e.g. a 25, 50, 75, 100, 105, 110, 115, 120, 125, 130, 135, 140, 145 or 150 continuous amino acid portion) compared to a naturally occurring PD-L1 polypeptide. In embodiments, the variants or homologs have about 90%, 95%, 96%, 97%, 98%, 99% or 100% amino acid sequence identity across the whole sequence or a portion of the sequence (e.g. about 25, 50, 75, 100, 105, 110, 115, 120, 125, 130, 135, 140, 145 or 150 continuous amino acid portion) compared to a naturally occurring PD-L1 polypeptide. In embodiments, PD-L1 is the protein as identified by the NCBI sequence reference GI: 30088843, homolog or functional fragment thereof. In embodiments, PD-L1 is the protein as identified by the NCBI sequence reference GI: 83287884, homolog or functional fragment thereof.

The term “STAT3” or “STAT3 protein” as provided herein includes any of the recombinant or naturally-occurring forms of the signal transducer and activator of transcription 3 protein (STAT3) or variants or homologs thereof that maintain STAT3 protein activity (e.g. within at least 50%, 80%, 90%, 95%, 96%, 97%, 98%, 99% or 100% activity compared to STAT3). In some aspects, the variants or homologs have at least 90%, 95%, 96%, 97%, 98%, 99% or 100% amino acid sequence identity across the whole sequence or a portion of the sequence (e.g. a 50, 100, 150, 200, 250, 300, 350, 400, 450, 500, 550, 600, 650, 700, 750, 800, 805, 810, 815, 820, 825, 830, 835, or 840 continuous amino acid portion) compared to a naturally occurring STAT3 polypeptide. In embodiments, the variants or homologs have about 90%, 95%, 96%, 97%, 98%, 99% or 100% amino acid sequence identity across the whole sequence or a portion of the sequence (e.g. about 50, 100, 150, or 200, 250, 300, 350, 400, 450, 500, 550, 600, 650, 700, 750, 800, 805, 810, 815, 820, 825, 830, 835, or 840 continuous amino acid portion) compared to a naturally occurring STAT3 polypeptide. In embodiments, STAT3 is the protein as identified by the NCBI sequence reference GI: 48429227, homolog or functional fragment thereof. In embodiments, STAT3 is the protein as identified by the NCBI sequence reference GI: 15680254, homolog or functional fragment thereof. In embodiments, STAT3 is the protein as identified by the NCBI sequence reference GI: 12653685, homolog or functional fragment thereof.

The term “TLR9” or “TLR9 protein”, also known as cluster of differentiation 289 (CD289) as provided herein includes any of the recombinant or naturally-occurring forms of the toll-like receptor 9 (TLR9) or variants or homologs thereof that maintain TLR9 protein activity (e.g. within at least 50%, 80%, 90%, 95%, 96%, 97%, 98%, 99% or 100% activity compared to TLR9). In some aspects, the variants or homologs have at least 90%, 95%, 96%, 97%, 98%, 99% or 100% amino acid sequence identity across the whole sequence or a portion of the sequence (e.g. a 50, 100, 150, 200, 250, 300, 350, 400, 450, 500, 550, 600, 650, 700, 750, 800, 850, 900, 950, 1000, 1005, 1010, 1015, 1020, 1025, 1030, 1035, 1040, 1045, 1050, 1055, 1060, 1065, 1070, 1075, 1080, 1085, 1090, 1095 or 1100 continuous amino acid portion) compared to a naturally occurring TLR9 polypeptide. In embodiments, the variants or homologs have about 90%, 95%, 96%, 97%, 98%, 99% or 100% amino acid sequence identity across the whole sequence or a portion of the sequence (e.g. about 50, 100, 150, 200, 250, 300, 350, 400, 450, 500, 550, 600, 650, 700, 750, 800, 850, 900, 950, 1000, 1005, 1010, 1015, 1020, 1025, 1030, 1035, 1040, 1045, 1050, 1055, 1060, 1065, 1070, 1075, 1080, 1085, 1090, 1095 or 1100 continuous amino acid portion) compared to a naturally occurring TLR9 polypeptide. In embodiments, TLR9 is the protein as identified by the NCBI sequence reference GI: 11761321, homolog or functional fragment thereof. In embodiments, TLR9 is the protein as identified by the NCBI sequence reference GI: 8575529, homolog or functional fragment thereof. In embodiments, TLR9 is the protein as identified by the NCBI sequence reference GI: 20140872, homolog or functional fragment thereof.

The term “PARP” or “PARP protein” as provided herein includes any of the recombinant or naturally-occurring forms of the poly(ADP-ribose) polymerase (PARP) or variants or homologs thereof that maintain PARP activity (e.g. within at least 50%, 80%, 90%, 95%, 96%, 97%, 98%, 99% or 100% activity compared to PARP). In some aspects, the variants or homologs have at least 90%, 95%, 96%, 97%, 98%, 99% or 100% amino acid sequence identity across the whole sequence or a portion of the sequence (e.g. a 50, 100, 150, 200, 250, 300, 350, 400, 450, 500, 550, 600, 650, 700, 750, 800, 850, 900, 950, 1000, 1005, 1010, 1015, 1020, 1025, 1030, 1035, 1040, 1045, 1050, 1055, 1060, 1065, 1070, 1075, 1080, 1085, 1090, 1095 or 1100 continuous amino acid portion) compared to a naturally occurring PARP. In embodiments, the variants or homologs have about 90%, 95%, 96%, 97%, 98%, 99% or 100% amino acid sequence identity across the whole sequence or a portion of the sequence (e.g. about 50, 100, 150, 200, 250, 300, 350, 400, 450, 500, 550, 600, 650, 700, 750, 800, 850, 900, 950, 1000, 1005, 1010, 1015, 1020, 1025, 1030, 1035, 1040, 1045, 1050, 1055, 1060, 1065, 1070, 1075, 1080, 1085, 1090, 1095 or 1100 continuous amino acid portion) compared to a naturally occurring PARP. In embodiments, PARP is the protein as identified by the NCBI sequence reference GI: 22902366, homolog or functional fragment thereof. In embodiments, PARP is the protein as identified by the NCBI sequence reference GI: 156523968, homolog or functional fragment thereof.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cunateous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound (e.g., an anti-STAT3-Toll-like receptor 9 (TLR9)-binding conjugate) as described herein (including embodiments and examples). In some embodiments, a control is the measurement of the expression level of a gene in the absence of a compound (e.g., an anti-STAT3-Toll-like receptor 9 (TLR9)-binding conjugate) as described herein (including embodiments and examples).

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

An “anticancer agent” as used herein refers to a molecule (e.g. compound, peptide, protein, nucleic acid) used to treat cancer through destruction or inhibition of cancer cells or tissues. Anticancer agents may be selective for certain cancers or certain tissues. In embodiments, anticancer agents herein may include epigenetic inhibitors and multi-kinase inhibitors.

“Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RH retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-Pseudomonas exotoxin conjugate, etc.), radioimmunotherapy (e.g., anti-CD20 monoclonal antibody conjugated to ¹¹¹In, ⁹⁰Y or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

A “multi-kinase inhibitor” is a small molecule inhibitor of at least one protein kinase, including tyrosine protein kinases and serine/threonine kinases. A multi-kinase inhibitor may include a single kinase inhibitor. Multi-kinase inhibitors may block phosphorylation. Multi-kinases inhibitors may act as covalent modifiers of protein kinases. Multi-kinase inhibitors may bind to the kinase active site or to a secondary or tertiary site inhibiting protein kinase activity. A multi-kinase inhibitor may be an anti-cancer multi-kinase inhibitor. Exemplary anti-cancer multi-kinase inhibitors include, but are not limited to, dasatinib, sunitinib, erlotinib, bevacizumab, vatalanib, vemurafenib, vandetanib, cabozantinib, ponatinib, axitinib, ruxolitinib, regorafenib, crizotinib, bosutinib, cetuximab, gefitinib, imatinib, lapatinib, lenvatinib, mubritinib, nilotinib, panitumumab, pazopanib, trastuzumab, or sorafenib.

As defined herein the terms “immune checkpoint”, “immune checkpoint protein” or “checkpoint protein” may be used interchangeably and refer to molecules capable of modulating the duration and amplitude of physiological immune responses. Immune checkpoint molecules may stimulate (increase) an immune response. In embodiments, the checkpoint protein is a cellular receptor. Examples, of stimulatory checkpoint molecules include, but are not limited to, members of the tumor necrosis factor (TNF) receptor superfamily (e.g. CD27, CD40, OX40, glucocorticoid-induced TNFR family related gene (GITR), and CD137), members of the B7-CD28 superfamily (e.g. CD28 itself and Inducible T-cell costimulator (ICOS)). Alternatively, immune checkpoint molecules may inhibit (decrease) an immune response. Examples of inhibitory checkpoint molecules include, but are not limited to, adenosine A2A receptor (A2AR), B7-H3, B7-H4, BTLA, CTLA-4, indoleamine 2,3-dioxygenase (IDO), killer immunoglobulin-like receptors (KIR), LAG3, PD-1, TIM-3, and V-domain immunoglobulin suppressor of T-cell activation (VISTA) protein.

As defined herein, the term “activation”, “activate”, “activating”, “activator” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

Likewise, an “immune checkpoint inhibitor” or “checkpoint inhibitor” as provided herein refers to a substance (e.g., an antibody or fragment thereof, a small molecule) that is capable of inhibiting, negatively affecting (e.g., decreasing) the activity or function of a checkpoint protein (e.g., decreasing expression or decreasing the activity of a checkpoint protein) relative to the activity or function of the checkpoint protein in the absence of the inhibitor. The checkpoint inhibitor may at least in part, partially or totally block stimulation, decrease, prevent, or delay activation, or inactivate, desensitize, or down-regulate signal transduction or enzymatic activity or the amount of a checkpoint protein. A “checkpoint inhibitor” may inhibit a checkpoint protein, e.g., by binding, partially or totally blocking, decreasing, preventing, delaying, inactivating, desensitizing, or down-regulating activity of the checkpoint protein. The inhibited expression or activity of the checkpoint protein can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control. In certain instances, the inhibitor reduces the activity of the checkpoint protein from an indirect or direct interaction. In embodiments, the checkpoint inhibitor is an antibody. In embodiments, the checkpoint inhibitor is an anti-CTLA4 antibody, an anti-PD-1 antibody or an anti-PD-L1 antibody. In embodiments, the checkpoint inhibitor is an antibody fragment. In embodiments, the checkpoint inhibitor is an antibody variant. In embodiments, the checkpoint inhibitor is a scFv. Non-limiting examples of checkpoint inhibitors include ipilimumab, pembrolizumab, nivolumab, talimogene laherparepvec, durvalumab, daclizumab, avelumab, and atezolizumab. In embodiments, the checkpoint inhibitor is ipilimumab. In embodiments, the checkpoint inhibitor is pembrolizumab. In embodiments, the checkpoint inhibitor is nivolumab. In embodiments, the checkpoint inhibitor is talimogene. In embodiments, the checkpoint inhibitor is laherparepvec. In embodiments, the checkpoint inhibitor is durvalumab. In embodiments, the checkpoint inhibitor is daclizumab. In embodiments, the checkpoint inhibitor is avelumab. In embodiments, the checkpoint inhibitor is atezolizumab.

A “poly ADP ribose polymerase inhibitor” or “PARP inhibitor” as provided herein refers to a substance capable of detectably lowering expression of or activity level of PARP compared to a control. The inhibited expression or activity of PARP can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control. In certain instances, the inhibitor reduces the activity of Poly (ADP-ribose) polymerase from an indirect or direct interaction.

“S elective” or “selectivity” or the like of a compound refers to the compound's ability to discriminate between molecular targets (e.g. a compound having selectivity toward STAT3, CTLA4 or PARP activity).

“Specific”, “specifically”, “specificity”, or the like of a compound refers to the compound's ability to cause a particular action, such as inhibition, to a particular molecular target with minimal or no action to other proteins in the cell (e.g. a compound having specificity towards STAT3, CTLA4 or PARP displays inhibition of the activity of STAT3, CTLA4 or PARP, whereas the same compound displays little-to-no inhibition of other STATs or checkpoint proteins).

The terms “immune response” and the like refer, in the usual and customary sense, to a response by an organism that protects against disease. The response can be mounted by the innate immune system or by the adaptive immune system, as well known in the art.

The terms “modulating immune response” and the like refer to a change in the immune response of a subject as a consequence of administration of an agent, e.g., a compound as disclosed herein, including embodiments thereof. Accordingly, an immune response can be activated or deactivated as a consequence of administration of an agent, e.g., a compound as disclosed herein, including embodiments thereof.

“B Cells” or “B lymphocytes” refer to their standard use in the art. B cells are lymphocytes, a type of white blood cell (leukocyte), that develops into a plasma cell (a “mature B cell”), which produces antibodies. An “immature B cell” is a cell that can develop into a mature B cell. Generally, pro-B cells undergo immunoglobulin heavy chain rearrangement to become pro B pre B cells, and further undergo immunoglobulin light chain rearrangement to become an immature B cells. Immature B cells include T1 and T2 B cells.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

A “memory T cell” is a T cell that has previously encountered and responded to its cognate antigen during prior infection, encounter with cancer or previous vaccination. At a second encounter with its cognate antigen memory T cells can reproduce (divide) to mount a faster and stronger immune response than the first time the immune system responded to the pathogen.

A “regulatory T cell” or “suppressor T cell” is a lymphocyte which modulates the immune system, maintains tolerance to self-antigens, and prevents autoimmune disease.

By “effective dose or amount” herein is meant a dose that produces effects for which it is administered. By “combined effective dose or amount” herein is meant a dose of two or more agents administered concomitantly (e.g., as a mixture), separately but simultaneously (e.g., via separate intravenous lines) or sequentially (e.g., one agent is administered first followed by administration of the second agent), that produces effects for which it is administered. For example, therapeutically effective amount or combined effect amount includes that amount of an agent or combination of agents sufficient to reduce or ameliorate one or more symptoms of a disease or disorder. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)).

A “synergistic amount” as used herein refers to the sum of a first amount (e.g., an amount of a compound provided herein) and a second amount (e.g., a therapeutic agent) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of the compound administered in combination where the measured effect is greater than the sum of the individual effects of each of the compounds provided herein administered alone as a single agent.

In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the conjugate (anti-STAT3-Toll-like receptor 9 (TLR9)-binding conjugate) provided herein when used separately from the checkpoint inhibitor or PARP inhibitor. In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the checkpoint inhibitor or the PARP inhibitor when used separately from the conjugate (anti-STAT3-Toll-like receptor 9 (TLR9)-binding conjugate) provided herein.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The term “vaccine” refers to a composition that can provide active acquired immunity to and/or therapeutic effect (e.g. treatment) of a particular disease or a pathogen. A vaccine typically contains one or more agents that can induce an immune response in a subject against a pathogen or disease, i.e. a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g. preventing or ameliorating the effects of a future infection by any natural or pathogen, or of an anticipated occurrence of cancer in a predisposed subject) or therapeutic (e.g., treating cancer in a subject who has been diagnosed with the cancer). The administration of vaccines is referred to vaccination. In some examples, a vaccine composition can provide nucleic acid, e.g. mRNA that encodes antigenic molecules (e.g. peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g. one or more peptides that are known to be expressed in the pathogen (e.g. pathogenic bacterium or virus). In the context of cancer vaccine, the vaccine composition can provide mRNA encoding certain peptides that are associated with cancer, e.g. peptides that are substantially exclusively or highly expressed in cancer cells as compared to normal cells. The subject, after vaccination with the cancer vaccine composition, can have immunity against the peptides that are associated with cancer and kill the cancer cells with specificity.

The term “immune response” used herein encompasses, but is not limited to, an “adaptive immune response”, also known as an “acquired immune response” in which adaptive immunity elicits immunological memory after an initial response to a specific pathogen or a specific type of cells that is targeted by the immune response, and leads to an enhanced response to that target on subsequent encounters. The induction of immunological memory can provide the basis of vaccination.

The term “immunogenic” or “antigenic” refers to a compound or composition that induces an immune response, e.g., cytotoxic T lymphocyte (CTL) response, a B cell response (for example, production of antibodies that specifically bind the epitope), an NK cell response or any combinations thereof, when administered to an immunocompetent subject. Thus, an immunogenic or antigenic composition is a composition capable of eliciting an immune response in an immunocompetent subject. For example, an immunogenic or antigenic composition can include one or more immunogenic epitopes associated with a pathogen or a specific type of cells that is targeted by the immune response. In addition, an immunogenic composition can include isolated nucleic acid constructs (such as DNA or RNA) that encode one or more immunogenic epitopes of the antigenic polypeptide that can be used to express the epitope(s) (and thus be used to elicit an immune response against this polypeptide or a related polypeptide associated with the targeted pathogen or type of cells).

The term “EC50” or “half maximal effective concentration” as used herein refers to the concentration of a molecule (e.g., antibody, chimeric antigen receptor or bispecific antibody) capable of inducing a response which is halfway between the baseline response and the maximum response after a specified exposure time. In embodiments, the EC50 is the concentration of a molecule (e.g., antibody, chimeric antigen receptor or bispecific antibody) that produces 50% of the maximal possible effect of that molecule.

### Methods of Treating Cancer

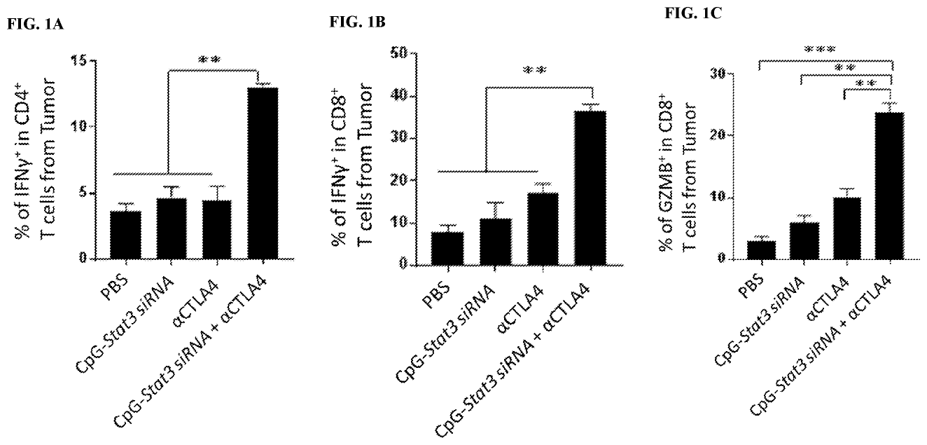

Provided herein are, inter alia, methods and compositions for the treatment of cancer. The methods include administering a combination of an anti-STAT3-Toll-like receptor 9 (TLR9)-binding conjugate and a checkpoint inhibitor. Applicants show that administration of a combined synergistic amount of the anti-STAT3-Toll-like receptor 9 (TLR9)-binding conjugate and the checkpoint inhibitor results in surprisingly increased anti-tumor efficacy and CD8 T cell activity (e.g., FIGS. 1-3). Applicants further show that TLR9 is expressed on T cell lymphoma cells and the conjugates provided herein including embodiments thereof are, inter alia, useful to target T cell lymphoma cells.

Thus, in one aspect is provided a method of treating cancer in a subject in need thereof. The method includes administering to the subject an effective amount of an anti-STAT3-Toll-like receptor 9 (TLR9)-binding conjugate and an effective amount of a checkpoint inhibitor. In one aspect is provided a method of treating cancer in a subject in need thereof. The method includes administering to the subject an anti-STAT3-Toll-like receptor 9 (TLR9)-binding conjugate and a checkpoint inhibitor in a combined effective amount. The anti-STAT3-Toll-like receptor 9 (TLR9)-binding conjugate provided herein is a conjugate including a STAT-3 inhibitory moiety bound to a TLR9-binding moiety through a chemical linker, wherein the TLR9-binding moiety is able to bind TLR9 and the STAT-3 inhibitory moiety is capable of detectably inhibiting STAT3 protein expression or STAT3 protein activity. In embodiments, the STAT-3 inhibitory moiety is covalently bound to a TLR9-binding moiety. In embodiments, the STAT-3 inhibitory moiety is non-covalently bound to a TLR9-binding moiety.

In embodiments, the TLR9-binding moiety is a peptide moiety. In embodiments, the TLR9-binding moiety is a nucleic acid moiety. In embodiments, the TLR9-binding moiety is a CpG moiety. A CpG moiety as referred to herein may include a nucleic acid referred to as “CpG oligodeoxynucleotide (ODN)”, “CpG nucleic acid sequence” or “GpC nucleic acid sequence.” A “CpG moiety” as provided herein may be a short single-stranded DNA molecule including unmethylated CpG dinucleotides in particular sequence contexts (CpG motifs).

The term “CpG motif” in a nucleic acid refers to a nucleic acid in which a 5” Cytosine (C) nucleotide is connected to a 3′ Guanine (G) nucleotide through a phosphodiester internucleotide linkage or a phosphodiester derivative internucleotide linkage. In embodiments, the term “CpG motif” in a nucleic acid refers to a nucleic acid in which a 5′ G nucleotide is connected to a 3′ C nucleotide through a phosphodiester internucleotide linkage or a phosphodiester derivative internucleotide linkage (aka a “GpC nucleic acid sequence”). In embodiments, a CpG motif includes a phosphodiester internucleotide linkage. In embodiments, a CpG motif includes a phosphodiester derivative internucleotide linkage. In embodiments, a CpG motif includes a phosphorothioate linkage.

The CpG moiety may include a backbone, wherein the backbone is partially or completely phosphorothioated. Wherein the CpG moiety is partially phosphorothioated the backbone includes phosphodiester (PO) and phosphorothiate (PS) internucleotide linkages. Wherein the CpG moiety is completely phosphorothioated the backbone includes phosphorothiate (PS) internucleotide linkages and no phosphodiester (PO) internucleotide linkages. In embodiments, the CpG moiety does not include phosphodiester (PO) internucleotide linkages. In embodiments, the CpG moiety includes a phosphodiester CpG-containing palindromic motif or “CpG” motif and/or a PS-modified 3′ poly-G string. In embodiments, the CpG moiety includes a CpG motif.

In embodiments, the CpG moiety may include a Class A phosphorothioated CpG oligodeoxynucleotide (CpG-ODN), Class B CpG ODN or Class C CpG ODN. In embodiments, the CpG moiety is a Class A phosphorothioated CpG oligodeoxynucleotide (CpG-ODN), Class B CpG ODN or Class C CpG ODN. As used herein, the term “Class A CpG ODN” or “A-class CpG ODN” or “D-type CpG ODN” or “Class A CpG DNA sequence” is used in accordance with its common meaning in the biological and chemical sciences and refers to a CpG motif including oligodeoxynucleotide including one or more of poly-G sequence at the 5′, 3′, or both ends; an internal palindrome sequence including CpG motif; or one or more phosphodiester derivatives linking deoxynucleotides. In embodiments, a Class A CpG ODN includes poly-G sequence at the 5′, 3′, or both ends; an internal palindrome sequence including CpG motif; and one or more phosphodiester derivatives linking deoxynucleotides. In embodiments, the phosphodiester derivative is phosphorothioate. Examples of Class A CpG ODNs include ODN D19, ODN 1585, ODN 2216, and ODN 2336.

As used herein, the term “Class B CpG ODN” or “B-class CpG ODN” or “K-type CpG ODN” or “Class B CpG DNA sequence” is used in accordance with its common meaning in the biological and chemical sciences and refers to a CpG motif including oligodeoxynucleotide including one or more of a 6mer motif including a CpG motif; phosphodiester derivatives linking all deoxynucleotides. In embodiments, a 6mer motif includes 5′-PuPyCGPyPu-3′ (SEQ ID NO:19; rycgyr), where Pu represents a purine containing nucleobase (e.g., A or G) and Py represents a pyrimidine containing nucleobase (e.g., T/U or C). In embodiments, a Class B CpG ODN includes one or more copies of a 6mer motif including a CpG motif and phosphodiester derivatives linking all deoxynucleotides. In embodiments, the phosphodiester derivative is phosphorothioate. In embodiments, a Class B CpG ODN includes one 6mer motif including a CpG motif. In embodiments, a Class B CpG ODN includes two copies of a 6mer motif including a CpG motif. In embodiments, a Class B CpG ODN includes three copies of a 6mer motif including a CpG motif. In embodiments, a Class B CpG ODN includes four copies of a 6mer motif including a CpG motif. Examples of Class B CpG ODNs include ODN 1668, ODN 1826, ODN 2006, and ODN 2007.

As used herein, the term “Class C CpG ODN” or “C-class CpG ODN” or “C-type CpG DNA sequence” is used in accordance with its common meaning in the biological and chemical sciences and refers to an oligodeoxynucleotide including a palindrome sequence including a CpG motif and phosphodiester derivatives (phosphorothioate) linking all deoxynucleotides. Examples of Class C CpG ODNs include ODN 2395 and ODN M362.

As used herein, the term “phosphorothioated oligodeoxynucleotide (ODN)” refers to a nucleic acid sequence, e.g., “CpG nucleic acid sequence” or “GpC nucleic acid sequence”, in which some or all the internucleotide linkages constitute a phosphorothioate linkage. In embodiments, phosphorothioated oligodeoxynucleotide (ODN) is 15 to 30 bases long, single-stranded, partly or completely phosphorothioated. The partly phosphorothioated ODN is an ODN in which 1, 2, 3, 4, 5, 6, 7, 8, 9, 10, 11, 12, 13, 14, 15, 16, 17, 18, 19, 20, 21, 22, 23, 24, 25, 26, 27, or 28, internucleotide linkages constitute a phosphorothioate linkage.

In embodiments, the CpG moiety is capable of inducing high IFN-α production from dendritic cells. In embodiments, the CpG moiety includes a phosphorothiate backbone with one or more CpG dinucleotides. In further embodiments, the CpG moiety is capable of activating B cells and TLR9-dependent NF-κB signaling.

In embodiments, the conjugate includes a nucleic acid sequence (CpG-ODN) having about 80%-100% (e.g., 80%, 85%, 90%, 91%, 92%, 93%, 94%, 95%, 96%, 97%, 98%, 99% or 100%) sequence identity with at least a 10 nucleobase continuous sequence of one of SEQ ID NOs:4-17, conjugated to a STAT-3 inhibitory moiety. In embodiments, the conjugate includes a nucleic acid sequence (CpG-ODN) having about 80%-100% (e.g., 80%, 85%, 90%, 91%, 92%, 93%, 94%, 95%, 96%, 97%, 98%, 99% or 100%) sequence identity with at least a 15 nucleobase continuous sequence of one of SEQ ID NOs:4-17, conjugated to a STAT-3 inhibitory moiety. In embodiments, the conjugate includes a nucleic acid sequence (CpG-ODN) having about 80%-100% (e.g., 80%, 85%, 90%, 91%, 92%, 93%, 94%, 95%, 96%, 97%, 98%, 99% or 100%) sequence identity with one of SEQ ID NOs:4-17, conjugated to a STAT-3 inhibitory moiety. In embodiments, the conjugate includes a nucleic acid sequence (CpG-ODN) having about 80%-100% (e.g., 80%, 85%, 90%, 91%, 92%, 93%, 94%, 95%, 96%, 97%, 98%, 99% or 100%) sequence identity with at least a 20 nucleobase continuous sequence of one of SEQ ID NOs:4-17, conjugated to a STAT-3 inhibitory moiety. In embodiments, the conjugate includes a nucleic acid sequence (CpG-ODN) having about 80%-100% (e.g., 80%, 85%, 90%, 91%, 92%, 93%, 94%, 95%, 96%, 97%, 98%, 99% or 100%) sequence identity with one of SEQ ID NOs:4-17, conjugated to a STAT-3 inhibitory moiety. In embodiments, the nucleic acid sequence (CpG-ODN) is 15 to 30 (e.g., 15, 16, 17, 18, 19, 20, 21, 22, 23, 24, 25, 26, 27, 28, 29, or 30) bases long, single-stranded, partly or completely phosphorothioated oligonucleotide

In embodiments, the conjugate includes a nucleic acid sequence (CpG-ODN) having 80%-100% (e.g., 80%, 85%, 90%, 91%, 92%, 93%, 94%, 95%, 96%, 97%, 98%, 99% or 100%) sequence identity with at least a 10 nucleobase continuous sequence of one of SEQ ID NOs:4-17, conjugated to a STAT-3 inhibitory moiety. In embodiments, the conjugate includes a nucleic acid sequence (CpG-ODN) having 80%-100% (e.g., 80%, 85%, 90%, 91%, 92%, 93%, 94%, 95%, 96%, 97%, 98%, 99% or 100%) sequence identity with at least a 15 nucleobase continuous sequence of one of SEQ ID NOs:4-17, conjugated to a STAT-3 inhibitory moiety. In embodiments, the conjugate includes a nucleic acid sequence (CpG-ODN) having 80%-100% (e.g., 80%, 85%, 90%, 91%, 92%, 93%, 94%, 95%, 96%, 97%, 98%, 99% or 100%) sequence identity with one of SEQ ID NOs:4-17, conjugated to a STAT-3 inhibitory moiety. In embodiments, the conjugate includes a nucleic acid sequence (CpG-ODN) having 80%-100% (e.g., 80%, 85%, 90%, 91%, 92%, 93%, 94%, 95%, 96%, 97%, 98%, 99% or 100%) sequence identity with at least a 20 nucleobase continuous sequence of one of SEQ ID NOs:4-17, conjugated to a STAT-3 inhibitory moiety. In embodiments, the conjugate includes a nucleic acid sequence (CpG-ODN) having 80%-100% (e.g., 80%, 85%, 90%, 91%, 92%, 93%, 94%, 95%, 96%, 97%, 98%, 99% or 100%) sequence identity with one of SEQ ID NOs:4-17, conjugated to a STAT-3 inhibitory moiety. In embodiments, the nucleic acid sequence (CpG-ODN) is 15 to 30 (e.g., 15, 16, 17, 18, 19, 20, 21, 22, 23, 24, 25, 26, 27, 28, 29, or 30) bases long, single-stranded, partly or completely phosphorothioated oligonucleotide

In embodiments, the conjugate includes a nucleic acid sequence (CpG-ODN) having about 80-85%, about 85-90%, about 90-95%, about 95%-100% sequence identity with at least a 15 nucleobase continuous sequence of one of SEQ ID NOs:4-17, and is conjugated to a STAT-3 inhibitory moiety. In embodiments, the conjugate includes a nucleic acid sequence (CpG-ODN) having about 80-85%, about 85-90%, about 90-95%, about 95%-100% sequence identity with one of SEQ ID NOs:4-17, conjugated to a STAT-3 inhibitory moiety. In embodiments, the nucleic acid sequence (CpG-ODN) is a 15 to 30 (e.g., 15, 16, 17, 18, 19, 20, 21, 22, 23, 24, 25, 26, 27, 28, 29, or 30) bases long, single-stranded, partly or completely phosphorothioated oligonucleotide.

The nucleic acid sequences (CpG-ODN) of SEQ ID NOs:4-17 are listed in Table 1.

TABLE 1
Compound and component sequences.

| | | SEQ |
|---|---|---|
| | SEQUENCE 5′-3′ | ID |
| NAME | (* = phosphorothioate linkage) | NO: |
| CpG(A)- | G*G*TGCATCGATGCAGG*G*G*G*G | 1/4 |
| ODN | | |
| GpC(A)- | G*G*T GCA TGC ATG CAG G*G*G*G*G | 5 |
| ODN | | |
| D19-PS | G*G*T*G*C*A*T*C*G*A*T*G*C*A*G*G*G*G*G*G | 6 |
| CpG(B)- | T*C*C*A*T*G*A*C*G*T*T*C*C*T*G*A*T*G*C*T | 7 |
| ODN | | |
| ODN 1585 | G*G*GGTCAACGTTGAG*G*G*G*G*G or | 8 |
| | G*GGGTCAACGTTGAG*G*G*G*G*G | |
| ODN 2216 | G*G*GGGACGA:TCGTCG*G*G*G*G*G or | 9 |
| | G*GGGGACGA:TCGTCG*G*G*G*G*G | |
| ODN D19 | G*G*TGCATCGATGCAGG*G*G*G*G or | 10 |
| | G*GTGCATCGATGCAGG*G*G*G*G | |
| ODN 2336 | G*G*G*GACGACGTCGTGG*G*G*G*G*G or | 11 |
| | G*G*GGACGACGTCGTGG*G*G*G*G*G | |
| ODN 1668 | T*C*C*A*T*G*A*C*G*T*T*C*C*T*G*A*T*G*C*T | 12 |
| ODN 1826 | T*C*C*A*T*G*A*C*G*T*T*C*C*T*G*A*C*G*T*T | 13 |
| ODN 2006 | T*C*G*T*C*G*T*T*T*T*G*T*C*G*T*T*T*T*G*T*C*G*T*T | 14 |
| (ODN7909) | | |
| ODN 2007 | T*C*G*T*C*G*T*T*G*T*C*G*T*T*T*T*G*T*C*G*T*T | 15 |
| ODN 2395 | T*C*G*T*C*G*T*T*T*T*C*G*G*C*G*C*G*C*G*C*C*G | 16 |
| ODN M362 | T*C*G*T*C*G*T*C*G*T*T*C*G*A*A*C*G*A*C*G*T*T*G*A* | 17 |
| | T | |

In embodiments, the STAT-3 inhibitory moiety is a peptide moiety. A “STAT-3 inhibitory moiety” as provided herein is a peptide or nucleic acid moiety capable of detectably decreasing the expression or activity of a STAT3 gene or STAT3 protein. The STAT-3 inhibitory moiety may decrease expression or activity of a STAT3 gene or STAT3 protein by 10%, 20%, 30%, 40%, 50%, 60%, 70%, 80%, 90% or more in comparison to a control in the absence of the STAT-3 inhibitory moiety. In certain instances, expression or activity of a STAT3 gene or STAT3 protein is 1.5-fold, 2-fold, 3-fold, 4-fold, 5-fold, 10-fold or lower than the expression or activity in the absence of the STAT-3 inhibitory moiety.

In embodiments, the STAT-3 inhibitory moiety is a small molecule. In embodiments, the STAT-3 inhibitory moiety is a nucleic acid moiety. In embodiments, the STAT-3 inhibitory moiety is an anti-sense nucleic acid moiety. In embodiments, the STAT-3 inhibitory moiety is an anti-sense STAT3 nucleic acid moiety.

An “anti-sense STAT3 nucleic acid moiety” or “anti-sense STAT3 nucleic acid” as provided herein is s a nucleic acid (e.g., DNA or RNA molecule) that is complementary to at least a portion of a STAT3 nucleic acid in a cell (e.g., DNA, mRNA). The anti-sense STAT3 nucleic acid moiety or anti-sense STAT3 nucleic acid is capable of reducing transcription of the STAT3 nucleic acid (e.g. mRNA from DNA), reducing the translation of the STAT3 nucleic acid (e.g. mRNA), altering transcript splicing (e.g. single stranded morpholino oligo), or interfering with the endogenous activity of the STAT3 nucleic acid. The anti-sense STAT3 nucleic acid moiety or anti-sense STAT3 nucleic acid may be a synthetic antisense nucleic acid (e.g. oligonucleotides) with a length of about 15 and 25 nucleotides. The anti-sense STAT3 nucleic acid moiety or anti-sense STAT3 nucleic acid is capable of hybridizing to (e.g. selectively hybridizing to) a STAT3 nucleic acid (e.g., STAT3 gene, STAT3 promoter, STAT3 mRNA). In embodiments, the anti-sense STAT3 nucleic acid moiety or anti-sense STAT3 nucleic acid hybridizes to the STAT3 nucleic acid in vitro. In embodiments, the anti-sense STAT3 nucleic acid moiety or anti-sense STAT3 nucleic acid hybridizes to the STAT3 nucleic acid in a cell. In embodiments, the anti-sense STAT3 nucleic acid moiety or anti-sense STAT3 nucleic acid hybridizes to the STAT3 nucleic acid in an organism. In embodiments, the anti-sense STAT3 nucleic acid moiety or anti-sense STAT3 nucleic acid hybridizes to the STAT3 nucleic acid under physiological conditions. The anti-sense STAT3 nucleic acid moiety or anti-sense STAT3 nucleic acid may comprise naturally occurring nucleotides or modified nucleotides such as, e.g., phosphorothioate, methylphosphonate, and -anomeric sugar-phosphate, backbone-modified nucleotides.

In embodiments, the STAT-3 inhibitory moiety is an anti-STAT3 siRNA. The anti-STAT3 siRNA may be a synthetic antisense nucleic acid (e.g. oligonucleotides) with a length of about 15 and 25 nucleotides. The anti-STAT3 siRNA is capable of hybridizing to (e.g. selectively hybridizing to) a STAT3 nucleic acid (e.g., STAT3 gene, STAT3 promoter, STAT3 mRNA). In embodiments, the anti-STAT3 siRNA hybridizes to the STAT3 nucleic acid in vitro. In embodiments, the anti-STAT3 siRNA hybridizes to the STAT3 nucleic acid in a cell. In embodiments, the anti-STAT3 siRNA hybridizes to the STAT3 nucleic acid in an organism. In embodiments, the anti-STAT3 siRNA hybridizes to the STAT3 nucleic acid under physiological conditions. The anti-STAT3 siRNA may comprise naturally occurring nucleotides or modified nucleotides such as, e.g., phosphorothioate, methylphosphonate, and -anomeric sugar-phosphate, backbone-modified nucleotides. In some embodiments, the degree of complementarity between an anti-STAT3 siRNA and its corresponding STAT3 RNA sequence, when optimally aligned using a suitable alignment algorithm, is about or more than about 50%, 60%, 75%, 80%, 85%, 90%, 91%, 92%, 93%, 94%, 95%, 96%, 97%, 98%, 99%, or 100%. In embodiments, the anti-STAT3 siRNA has at least 80%, 85%, 90%, 95%, 96%, 97%, 98%, 99% or 100% sequence identity with the perfectly complementary sequence of the STAT3 RNA sequence.

Thus, in embodiments, the anti-STAT3-TLR9 binding conjugate includes a CpG moiety bound to an anti-STAT3 siRNA through a chemical linker. In embodiments, the anti-STAT3-TLR9 binding conjugate includes a CpG ODN bound to an anti-STAT3 siRNA through a chemical linker.

In embodiments, the anti-STAT3 siRNA is a double-stranded RNA. Thus, in embodiments, the anti-STAT3 siRNA includes an antisense strand hybridized to a sense strand. In embodiments, the anti-STAT3 siRNA includes a sense strand having the sequence 5′ GGAAGCUGCAGAAAGAUACGACUGA 3′ (SEQ ID NO:3) hybridized to the antisense strand of SEQ ID NO:2. In embodiments, the anti-STAT3 siRNA includes a sense strand having 80%, 85%, 90%, 95%, 96%, 97%, 98%, 99% or 100% sequence identity with sequence 5′ GGAAGCUGCAGAAAGAUACGACUGA 3′ (SEQ ID NO:3) hybridized to the antisense strand having 80%, 85%, 90%, 95%, 96%, 97%, 98%, 99% or 100% sequence identity with SEQ ID NO:2.

A “chemical linker,” as provided herein, is a covalent linker, a non-covalent linker, a peptide or peptidyl linker (a linker including a peptide moiety), a cleavable peptide linker, a substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene or any combination thereof. Thus, a chemical linker as provided herein may include a plurality of chemical moieties, wherein each of the plurality of chemical moieties is chemically different. Alternatively, the chemical linker may be a non-covalent linker. Examples of non-covalent linkers include without limitation, ionic bonds, hydrogen bonds, halogen bonds, van der Waals interactions (e.g. dipole-dipole, dipole-induced dipole, London dispersion), ring stacking (pi effects), and hydrophobic interactions. In embodiments, a chemical linker is formed using conjugate chemistry including, but not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition).

The chemical linker as provided herein, may be —O—, —S—, —C(O)—, —C(O)O—, —C(O)NH—, —S(O)₂NH—, —NH—, —NHC(O)NH—, substituted (e.g., substituted with a substituent group, a size-limited substituent or a lower substituent group) or unsubstituted alkylene, substituted (e.g., substituted with a substituent group, a size-limited substituent or a lower substituent group) or unsubstituted heteroalkylene, substituted (e.g., substituted with a substituent group, a size-limited substituent or a lower substituent group) or unsubstituted cycloalkylene, substituted (e.g., substituted with a substituent group, a size-limited substituent or a lower substituent group) or unsubstituted heterocycloalkylene, substituted (e.g., substituted with a substituent group, a size-limited substituent or a lower substituent group) or unsubstituted arylene or substituted (e.g., substituted with a substituent group, a size-limited substituent or a lower substituent group) or unsubstituted heteroarylene.

The chemical linker as provided herein, may be —O—, —S—, —C(O)—, —C(O)O—, —C(O)NH—, —S(O)₂NH—, —NH—, —NHC(O)NH—, substituted or unsubstituted (e.g., C₁-C₂₀, C₁-C₁₀, C₁-C₅) alkylene, substituted or unsubstituted (e.g., 2 to 20 membered, 2 to 10 membered, 2 to 5 membered) heteroalkylene, substituted or unsubstituted (e.g., C₃-C₈, C₃-C₆, C₃-C₅) cycloalkylene, substituted or unsubstituted (e.g., 3 to 8 membered, 3 to 6 membered, 3 to 5 membered) heterocycloalkylene, substituted or unsubstituted (e.g., C₆-C₁₀, C₆-C₈, C₆-C₅) arylene or substituted or unsubstituted (e.g., 5 to 10 membered, 5 to 8 membered, 5 to 6 membered) heteroarylene.

In embodiments, the anti-STAT3-TLR9 binding conjugate includes a CpG moiety bound to an anti-STAT3 siRNA through a covalent linker. The anti-STAT3-TLR9 binding conjugate may be any of the conjugates described in U.S. Pat. No. 8,748,405, which is hereby incorporated by reference in its entirety and for all purposes. In embodiments, the anti-STAT3-TLR9 binding conjugate has the structure of:

CpG moiety-linker-anti-STAT3 siRNA (I),

wherein the linker is a substituted or unsubstituted alkylene.

In embodiments, the linker is a substituted or unsubstituted C₁-C₁₀ alkylene. In embodiments, the linker is an unsubstituted C₁-C₁₀ alkylene. In embodiments, the linker is a substituted or unsubstituted C₂-C₁₀ alkylene. In embodiments, the linker is an unsubstituted C₂-C₁₀ alkylene. In embodiments, the linker is a substituted or unsubstituted C₃-C₁₀ alkylene. In embodiments, the linker is an unsubstituted C₃-C₁₀ alkylene. In embodiments, the linker is a substituted or unsubstituted C₄-C₁₀ alkylene. In embodiments, the linker is an unsubstituted C₄-C₁₀ alkylene. In embodiments, the linker is a substituted or unsubstituted C₅-C₁₀ alkylene. In embodiments, the linker is an unsubstituted C₅-C₁₀ alkylene. In embodiments, the linker is a substituted or unsubstituted C₆-C₁₀ alkylene. In embodiments, the linker is an unsubstituted C₆-C₁₀ alkylene. In embodiments, the linker is a substituted or unsubstituted C₇-C₁₀ alkylene. In embodiments, the linker is an unsubstituted C₇-C₁₀ alkylene. In embodiments, the linker is a substituted or unsubstituted C₈-C₁₀ alkylene. In embodiments, the linker is an unsubstituted C₈-C₁₀ alkylene.

In embodiments, the linker is a substituted or unsubstituted C₁ alkylene. In embodiments, the linker is an unsubstituted C₁ alkylene. In embodiments, the linker is a substituted or unsubstituted C₂ alkylene. In embodiments, the linker is an unsubstituted C₂ alkylene. In embodiments, the linker is a substituted or unsubstituted C₃ alkylene. In embodiments, the linker is an unsubstituted C₃ alkylene. In embodiments, the linker is a substituted or unsubstituted C₄ alkylene. In embodiments, the linker is an unsubstituted C₄ alkylene. In embodiments, the linker is a substituted or unsubstituted C₅ alkylene. In embodiments, the linker is an unsubstituted C₅ alkylene. In embodiments, the linker is a substituted or unsubstituted C₆ alkylene. In embodiments, the linker is an unsubstituted C₆ alkylene. In embodiments, the linker is a substituted or unsubstituted C₇ alkylene. In embodiments, the linker is an unsubstituted C₇ alkylene. In embodiments, the linker is a substituted or unsubstituted C₈ alkylene. In embodiments, the linker is an unsubstituted C₈ alkylene. In embodiments, the linker is a substituted or unsubstituted C₉ alkylene. In embodiments, the linker is an unsubstituted C₉ alkylene. In embodiments, the linker is a substituted or unsubstituted C₁₀ alkylene. In embodiments, the linker is an unsubstituted C₁₀ alkylene. In embodiments, the covalent linker is an unsubstituted C₆ alkylene. In embodiments, the covalent linker is an unsubstituted C₇ alkylene.

In embodiments, the anti-STAT3-TLR9 binding conjugate has the structure of:

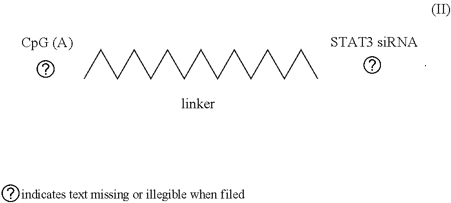

In formula (II), the sequence on the left of the linker has the sequence of SEQ ID NO:1, the top sequence to the right of the linker has the sequence of SEQ ID NO:2 and the bottom sequence to the right of the linker has the sequence of SEQ ID NO:3. The top sequence to the right of the linker having the sequence of SEQ ID NO:2 is also referred to herein as “antisense strand”, while the bottom sequence to the right of the linker having the sequence of SEQ ID NO:3 is referred to as “sense strand.” In formula (II) * indicates a phosphorothioate linkage, and ∧∧∧∧∧∧∧, indicates a chemical linker described herein. Any of the anti-STAT3 siRNAs described in U.S. Pat. No. 8,748,405, which is hereby incorporated by reference in its entirety and for all purposes, may be used for the conjugates of embodiments provided herein.

In embodiments, the linker represented by “xxxxx” or “∧∧∧∧∧∧∧,” is a bond, substituted (e.g. substituted with a substituent group, size-limited substituent group or lower substituent group) or unsubstituted alkylene, substituted (e.g. substituted with a substituent group, size-limited substituent group or lower substituent group) or unsubstituted heteroalkylene, substituted (e.g. substituted with a substituent group, size-limited substituent group or lower substituent group) or unsubstituted cycloalkylene, substituted (e.g. substituted with a substituent group, size-limited substituent group or lower substituent group) or unsubstituted heterocycloalkylene, substituted (e.g. substituted with a substituent group, size-limited substituent group or lower substituent group) or unsubstituted arylene, substituted (e.g. substituted with a substituent group, size-limited substituent group or lower substituent group) or unsubstituted cyclo-heteroalkylene or —(CH₂)ₙ, —PO₄—[(CH₂)ₙ—PO₄]_{z}—(CH₂)ₙ, in which the symbol n is an integer from 1 to 5 (e.g., 3) and the symbol z is an integer from 0 to 50 (e.g. from 0 to 25, 0 to 10, or 0 to 5). In embodiments, n is 3 and z is 0 to 5 or 1 to 5. In embodiments, n is 3 and z is 0 to 4 or 1 to 4. In embodiments, n is 3 and z is 0 to 3 or 1 to 3. In embodiments, n is 3 and z is 3. 2′OMe (2′-O-Methylnucleoside; Hydroxyl in 2′-position replaced with 2′-OMethyl); PS is phoshorothioation. One none-bridging oxygen replaced with sulfur; PS+3 represents three phosphates in the sequence modified, had one none-bridging oxygen replaced with sulfur; PS+5 represents five phosphates in the sequence modified, had one none-bridging oxygen replaced with sulfur.

For example, as shown below, in embodiments, nucleobases in the CpG moiety (e.g., phosphorothioated ODN) may include a phosphorothioate internucleotide linkage. A portion of such a phosphorothioated oligonucleotide is shown below.

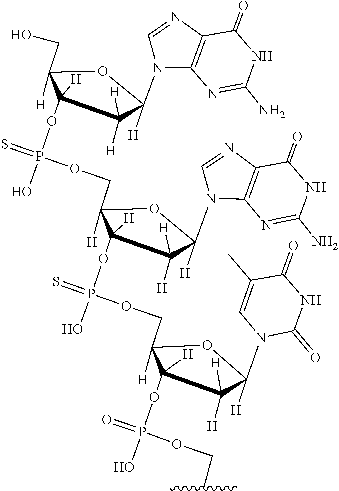

The linker may have the structure below, where the linker connects with the 3′ phosphate of the guanine on one end and the 5′ phosphate of the thymidine on the other end, and the nucleobases in the antisense strand may be modified with 2′OMe.

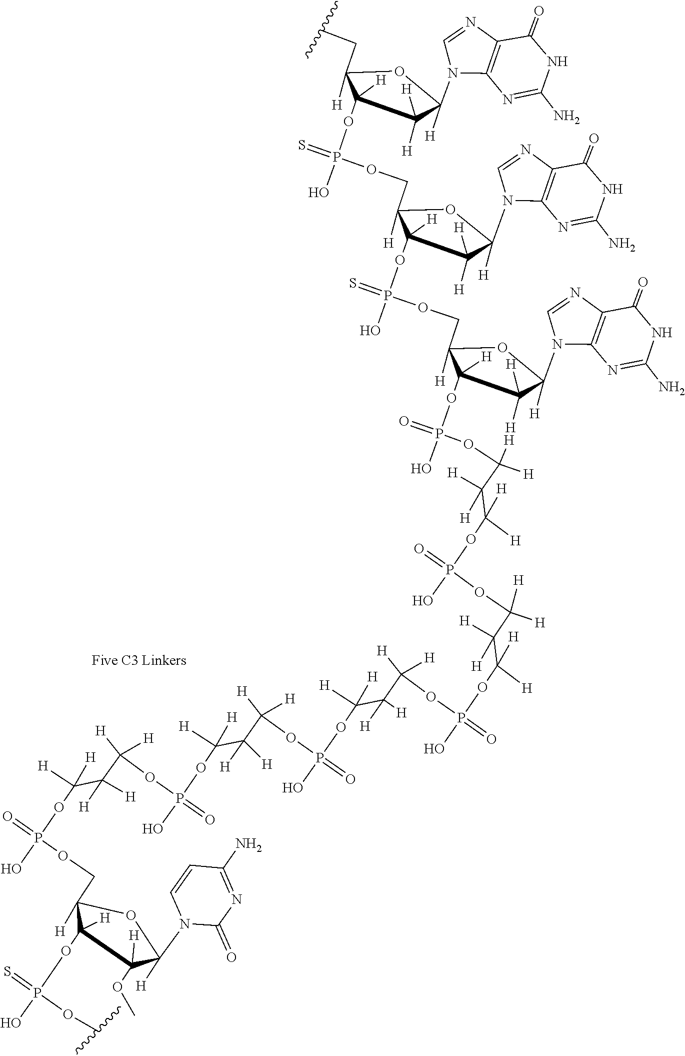

The above formula represents a portion of the CpG-ODN linked at the 3′-OH end with a (CH₂)₃ linker (also referred to herein as the C3 linker), which links to the 5′-phosphate of the anti-sense strand of the anti-STAT3 siRNA (SEQ ID NO:2) through another four C3 linkers. Thus, depicted above are five C3 linkers each consisting of three —CH₂— linkers (i.e. —(CH₂)₃—). Provided herein each C3 linker may also be referred to as “X” in the context of the conjugates provided herein.

The linker may be a bond, substituted (e.g. substituted with a substituent group, size-limited substituent group or lower substituent group) or unsubstituted alkylene, substituted (e.g. substituted with a substituent group, size-limited substituent group or lower substituent group) or unsubstituted heteroalkylene, substituted (e.g. substituted with a substituent group, size-limited substituent group or lower substituent group) or unsubstituted cycloalkylene, substituted (e.g. substituted with a substituent group, size-limited substituent group or lower substituent group) or unsubstituted heterocycloalkylene, substituted (e.g. substituted with a substituent group, size-limited substituent group or lower substituent group) or unsubstituted arylene, or substituted or unsubstituted heteroarylene.

In embodiments, the present disclosure includes a conjugate linking a phosphorothioated oligonucleotide of the present disclosure to an anti-STAT3 siRNA, where the conjugate has the sequence: 5′ G*G*T GCA TCG ATG CAGG*G*G*G*G (SEQ ID NO: 1) xxxxx UCAGUCGUAUCUUUCUGCAGCUUCCGU (SEQ ID NO:2) 3′, where * indicates a phosphorothioate linkage, and x indicates a linker described herein (e.g., —(CH₂)₃—). In further embodiments, the anti-STAT3 siRNA includes 5′ GGAAGCUGCAGAAAGAUACGACUGA 3′ (SEQ ID NO:3) hybridized to SEQ ID NO:2. In embodiments, the anti-STAT3 siRNA includes 5′ GGmAmAGmCmUGmCmAGmAmAmAGmAmUmAmCGACmUGA 3′ (SEQ ID NO:18) hybridized to SEQ ID NO:2, wherein mN indicates a 2′OMe, 2′F, LNA or an otherwise modified nucleotide.

In embodiments, the checkpoint inhibitor is an antibody or a small molecule. In embodiments, the checkpoint inhibitor is an antibody. In embodiments, the checkpoint inhibitor is a small molecule. In embodiments, the checkpoint inhibitor is an anti-CTLA4 antibody, an anti-PD-1 antibody or an anti-PD-L1 antibody. In embodiments, the checkpoint inhibitor is an anti-CTLA4 antibody. In embodiments, the checkpoint inhibitor is an anti-PD-1 antibody. In embodiments, the checkpoint inhibitor is an anti-PD-L1 antibody. In embodiments, the anti-PD-1 antibody is cemiplimab, pembrolizumab or nivolumab. In embodiments, the anti-PD-1 antibody is pembrolizumab. In embodiments, the anti-PD-1 antibody is nivolumab. In embodiments, the anti-PD-1 antibody is cemiplimab. In embodiments, the anti-PD-L1 antibody is atezolizumab, avelumab, or durvalumab. In embodiments, the anti-PD-L1 antibody is atezolizumab. In embodiments, the anti-PD-L1 antibody is avelumab. In embodiments, the anti-PD-L1 antibody is durvalumab. In embodiments, the anti-CTLA4 antibody is ipilimumab or tremelimumab. In embodiments, the anti-CTLA4 antibody is ipilimumab. In embodiments, the anti-CTLA4 antibody is tremelimumab.

In another aspect is provided a method of treating cancer in a subject in need thereof. The method includes administering to the subject an effective amount of an anti-STAT3-Toll-like receptor 9 (TLR9)-binding conjugate and an effective amount of a poly ADP ribose polymerase inhibitor (PARP inhibitor). In another aspect is provided a method of treating cancer in a subject in need thereof. The method includes administering to the subject an anti-STAT3-Toll-like receptor 9 (TLR9)-binding conjugate and a PARP inhibitor in a combined effective amount. In embodiments, the combined effective amount is a combined synergistic amount. In embodiments, the PARP inhibitor lowers expression of or activity level of PARP1, PARP2 or both. In embodiments, the PARP inhibitor lowers expression of or activity level of PARP1, PARP2, PARP3, PARP4 or any combination thereof. In embodiments, the PARP inhibitor lowers expression of or activity level of a specific PARP (e.g., PARP1) or of two or more homologs of PARP (e.g., PARP1, PARP2, PARP3, PARP4 etc.). A “PAPRP inhibitor” is a compound or small molecule that inhibits PARP e.g., by binding, partially or totally blocking stimulation of PARP, decrease, prevent, or delay activation of PARP, or inactivate, desensitize, or down-regulate signal transduction, gene expression or enzymatic activity of PARP. In embodiments, the PARP inhibitor inhibits PARP activity or expression PARP. In embodiments, the PARP inhibitor inhibits PARP activity or expression of PARP. In embodiments, the PARP inhibitor is a compound or a small molecule. In embodiments, the PARP inhibitor is an antibody. In embodiments, the PARP inhibitor is rucaparib, olaparib, niraparib, veliparib, talazoparib, vekauoarub, pamiparib, CEP 9722, E7016 (GPI-21016), BGB-290, INO-1001, MP-124, or LT-00673. In embodiments, the PARP inhibitor is rucaparib. In embodiments, the PARP inhibitor is olaparib. In embodiments, the PARP inhibitor is niraparib. In embodiments, the PARP inhibitor is veliparib. In embodiments, the PARP inhibitor is talazoparib. In embodiments, the PARP inhibitor is vekauoarub. In embodiments, the PARP inhibitor is pamiparib. In embodiments, the PARP inhibitor is CEP 9722. In embodiments, the PARP inhibitor is E7016 (GPI-21016). In embodiments, the PARP inhibitor is BGB-290. In embodiments, the PARP inhibitor is INO-1001. In embodiments, the PARP inhibitor is MP-124. In embodiments, the PARP inhibitor is LT-00673.

In embodiments, the method includes administering the anti-STAT3-TLR9-binding conjugate and the checkpoint inhibitor in a combined synergistic amount. In embodiments, the method includes administering the anti-STAT3-TLR9-binding conjugate and the checkpoint inhibitor in a combined therapeutically effective amount. In embodiments, the combined therapeutically effective amount is a combined synergistic amount. In embodiments, the anti-STAT3-TLR9-binding conjugate and the checkpoint inhibitor are administered simultaneously. In embodiments, the anti-STAT3-TLR9-binding conjugate and the checkpoint inhibitor are administered sequentially. In embodiments, the anti-STAT3-TLR9-binding conjugate is administered at a first time point and the checkpoint inhibitor is administered at a second time point, wherein the first time point precedes the second time point. In embodiments, the checkpoint inhibitor is administered at a first time point and the anti-STAT3-TLR9-binding conjugate is administered at a second time point, wherein the first time point precedes the second time point. In embodiments, the anti-STAT3-TLR9-binding conjugate and the checkpoint inhibitor are admixed prior to administration.

In embodiments, the method includes administering the anti-STAT3-TLR9-binding conjugate and the PARP inhibitor in a combined synergistic amount. In embodiments, the anti-STAT3-TLR9-binding conjugate and the PARP inhibitor are administered simultaneously. In embodiments, the anti-STAT3-TLR9-binding conjugate and the PARP inhibitor are administered sequentially. In embodiments, the anti-STAT3-TLR9-binding conjugate is administered at a first time point and the PARP inhibitor is administered at a second time point, wherein the first time point precedes the second time point. In embodiments, the PARP inhibitor is administered at a first time point and the anti-STAT3-TLR9-binding conjugate is administered at a second time point, wherein the first time point precedes the second time point. In embodiments, the anti-STAT3-TLR9-binding conjugate and the PARP inhibitor are admixed prior to administration.

In embodiments, the anti-STAT3-TLR9-binding conjugate is administered at an amount of about 10 mg to about 40 mg. In embodiments, the anti-STAT3-TLR9-binding conjugate is administered at an amount of about 15 mg to about 40 mg. In embodiments, the anti-STAT3-TLR9-binding conjugate is administered at an amount of about 20 mg to about 40 mg. In embodiments, the anti-STAT3-TLR9-binding conjugate is administered at an amount of about 25 mg to about 40 mg. In embodiments, the anti-STAT3-TLR9-binding conjugate is administered at an amount of about 30 mg to about 40 mg. In embodiments, the anti-STAT3-TLR9-binding conjugate is administered at an amount of about 35 mg to about 40 mg. In embodiments, the anti-STAT3-TLR9-binding conjugate is administered at an amount of about 10 mg to about 35 mg. In embodiments, the anti-STAT3-TLR9-binding conjugate is administered at an amount of about 10 mg to about 30 mg. In embodiments, the anti-STAT3-TLR9-binding conjugate is administered at an amount of about 10 mg to about 25 mg. In embodiments, the anti-STAT3-TLR9-binding conjugate is administered at an amount of about 10 mg to about 20 mg. In embodiments, the anti-STAT3-TLR9-binding conjugate is administered at an amount of about 10 mg to about 15 mg. In embodiments, the anti-STAT3-TLR9-binding conjugate is administered at an amount of about 10 mg, about 15 mg, about 20 mg, about 25 mg, about 30 mg, about 35 mg, or about 40 mg.

In embodiments, the anti-STAT3-TLR9-binding conjugate is administered at an amount of 10 mg to 40 mg. In embodiments, the anti-STAT3-TLR9-binding conjugate is administered at an amount of 15 mg to 40 mg. In embodiments, the anti-STAT3-TLR9-binding conjugate is administered at an amount of 20 mg to 40 mg. In embodiments, the anti-STAT3-TLR9-binding conjugate is administered at an amount of 25 mg to 40 mg. In embodiments, the anti-STAT3-TLR9-binding conjugate is administered at an amount of 30 mg to 40 mg. In embodiments, the anti-STAT3-TLR9-binding conjugate is administered at an amount of 35 mg to 40 mg. In embodiments, the anti-STAT3-TLR9-binding conjugate is administered at an amount of 10 mg to 35 mg. In embodiments, the anti-STAT3-TLR9-binding conjugate is administered at an amount of 10 mg to 30 mg. In embodiments, the anti-STAT3-TLR9-binding conjugate is administered at an amount of 10 mg to 25 mg. In embodiments, the anti-STAT3-TLR9-binding conjugate is administered at an amount of 10 mg to 20 mg. In embodiments, the anti-STAT3-TLR9-binding conjugate is administered at an amount of 10 mg to 15 mg. In embodiments, the anti-STAT3-TLR9-binding conjugate is administered at an amount of 10 mg, 15 mg, 20 mg, 25 mg, 30 mg, 35 mg, or 40 mg.

In embodiments, the anti-STAT3-TLR9-binding conjugate is administered at an amount of 10 mg/kg to 40 mg/kg. In embodiments, the anti-STAT3-TLR9-binding conjugate is administered at an amount of 15 mg/kg to 40 mg/kg. In embodiments, the anti-STAT3-TLR9-binding conjugate is administered at an amount of 20 mg/kg to 40 mg/kg. In embodiments, the anti-STAT3-TLR9-binding conjugate is administered at an amount of 25 mg/kg to 40 mg/kg. In embodiments, the anti-STAT3-TLR9-binding conjugate is administered at an amount of 30 mg/kg to 40 mg/kg. In embodiments, the anti-STAT3-TLR9-binding conjugate is administered at an amount of 35 mg/kg to 40 mg/kg. In embodiments, the anti-STAT3-TLR9-binding conjugate is administered at an amount of 10 mg/kg to 35 mg/kg. In embodiments, the anti-STAT3-TLR9-binding conjugate is administered at an amount of 10 mg/kg to 30 mg/kg. In embodiments, the anti-STAT3-TLR9-binding conjugate is administered at an amount of 10 mg/kg to 25 mg/kg. In embodiments, the anti-STAT3-TLR9-binding conjugate is administered at an amount of 10 mg/kg to 20 mg/kg. In embodiments, the anti-STAT3-TLR9-binding conjugate is administered at an amount of 10 mg/kg to 15 mg/kg. In embodiments, the anti-STAT3-TLR9-binding conjugate is administered at an amount of 10 mg/kg, 15 mg/kg, 20 mg/kg, 25 mg/kg, 30 mg/kg, 35 mg/kg, or 40 mg/kg.

In embodiments, the anti-STAT3-TLR9-binding conjugate is administered at an amount of 15 mg. In embodiments, the anti-STAT3-TLR9-binding conjugate is administered at an amount of about 15 mg. In embodiments, the anti-STAT3-TLR9-binding conjugate is administered at an amount of 30 mg. In embodiments, the anti-STAT3-TLR9-binding conjugate is administered at an amount of about 30 mg.

In embodiments, the anti-STAT3-TLR9-binding conjugate is administered at an amount of 0.1 mg/kg to 2 mg/kg. In embodiments, the anti-STAT3-TLR9-binding conjugate is administered at an amount of 0.2 mg/kg to 2 mg/kg. In embodiments, the anti-STAT3-TLR9-binding conjugate is administered at an amount of 0.3 mg/kg to 2 mg/kg. In embodiments, the anti-STAT3-TLR9-binding conjugate is administered at an amount of 0.4 mg/kg to 2 mg/kg. In embodiments, the anti-STAT3-TLR9-binding conjugate is administered at an amount of 0.5 mg/kg to 2 mg/kg. In embodiments, the anti-STAT3-TLR9-binding conjugate is administered at an amount of 0.6 mg/kg to 2 mg/kg. In embodiments, the anti-STAT3-TLR9-binding conjugate is administered at an amount of 0.7 mg/kg to 2 mg/kg. In embodiments, the anti-STAT3-TLR9-binding conjugate is administered at an amount of 0.8 mg/kg to 2 mg/kg. In embodiments, the anti-STAT3-TLR9-binding conjugate is administered at an amount of 0.9 mg/kg to 2 mg/kg. In embodiments, the anti-STAT3-TLR9-binding conjugate is administered at an amount of 1 mg/kg to 2 mg/kg. In embodiments, the anti-STAT3-TLR9-binding conjugate is administered at an amount of 1.2 mg/kg to 2 mg/kg. In embodiments, the anti-STAT3-TLR9-binding conjugate is administered at an amount of 1.3 mg/kg to 2 mg/kg. In embodiments, the anti-STAT3-TLR9-binding conjugate is administered at an amount of 1.4 mg/kg to 2 mg/kg. In embodiments, the anti-STAT3-TLR9-binding conjugate is administered at an amount of 1.5 mg/kg to 2 mg/kg. In embodiments, the anti-STAT3-TLR9-binding conjugate is administered at an amount of 1.6 mg/kg to 2 mg/kg. In embodiments, the anti-STAT3-TLR9-binding conjugate is administered at an amount of 1.7 mg/kg to 2 mg/kg. In embodiments, the anti-STAT3-TLR9-binding conjugate is administered at an amount of 1.8 mg/kg to 2 mg/kg. In embodiments, the anti-STAT3-TLR9-binding conjugate is administered at an amount of 1.9 mg/kg to 2 mg/kg. In embodiments, the anti-STAT3-TLR9-binding conjugate is administered at an amount of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, or 2 mg/kg. In embodiments, the anti-STAT3-TLR9-binding conjugate is administered at an amount of 0.56 mg/kg.

In embodiments, the anti-STAT3-TLR9-binding conjugate is administered at an amount of about 0.1 mg/kg to about 2 mg/kg. In embodiments, the anti-STAT3-TLR9-binding conjugate is administered at an amount of about 0.2 mg/kg to about 2 mg/kg. In embodiments, the anti-STAT3-TLR9-binding conjugate is administered at an amount of about 0.3 mg/kg to about 2 mg/kg. In embodiments, the anti-STAT3-TLR9-binding conjugate is administered at an amount of about 0.4 mg/kg to about 2 mg/kg. In embodiments, the anti-STAT3-TLR9-binding conjugate is administered at an amount of about 0.5 mg/kg to about 2 mg/kg. In embodiments, the anti-STAT3-TLR9-binding conjugate is administered at an amount of about 0.6 mg/kg to about 2 mg/kg. In embodiments, the anti-STAT3-TLR9-binding conjugate is administered at an amount of about 0.7 mg/kg to about 2 mg/kg. In embodiments, the anti-STAT3-TLR9-binding conjugate is administered at an amount of about 0.8 mg/kg to about 2 mg/kg. In embodiments, the anti-STAT3-TLR9-binding conjugate is administered at an amount of about 0.9 mg/kg to about 2 mg/kg. In embodiments, the anti-STAT3-TLR9-binding conjugate is administered at an amount of about 1 mg/kg to about 2 mg/kg. In embodiments, the anti-STAT3-TLR9-binding conjugate is administered at an amount of about 1.2 mg/kg to about 2 mg/kg. In embodiments, the anti-STAT3-TLR9-binding conjugate is administered at an amount of about 1.3 mg/kg to about 2 mg/kg. In embodiments, the anti-STAT3-TLR9-binding conjugate is administered at an amount of about 1.4 mg/kg to about 2 mg/kg. In embodiments, the anti-STAT3-TLR9-binding conjugate is administered at an amount of about 1.5 mg/kg to about 2 mg/kg. In embodiments, the anti-STAT3-TLR9-binding conjugate is administered at an amount of about 1.6 mg/kg to about 2 mg/kg. In embodiments, the anti-STAT3-TLR9-binding conjugate is administered at an amount of about 1.7 mg/kg to about 2 mg/kg. In embodiments, the anti-STAT3-TLR9-binding conjugate is administered at an amount of about 1.8 mg/kg to about 2 mg/kg. In embodiments, the anti-STAT3-TLR9-binding conjugate is administered at an amount of about 1.9 mg/kg to about 2 mg/kg. In embodiments, the anti-STAT3-TLR9-binding conjugate is administered at an amount of about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, or 2 mg/kg. In embodiments, the anti-STAT3-TLR9-binding conjugate is administered at an amount of about 0.56 mg/kg.

In embodiments, the checkpoint inhibitor is administered at an amount of about 0.1 to about 5 mg/kg. In embodiments, the checkpoint inhibitor is administered at an amount of 0.1 to 5 mg/kg. In embodiments, the checkpoint inhibitor is administered at an amount of 0.1, 1, 3, or 5 mg/kg. In embodiments, the checkpoint inhibitor is administered at an amount of 0.1 mg/kg. In embodiments, the checkpoint inhibitor is administered at an amount of 1 mg/kg. In embodiments, the checkpoint inhibitor is administered at an amount of 3 mg/kg. In embodiments, the checkpoint inhibitor is administered at an amount of 5 mg/kg. In embodiments, the checkpoint inhibitor is administered at an amount of about 0.1 mg/kg. In embodiments, the checkpoint inhibitor is administered at an amount of about 1 mg/kg. In embodiments, the checkpoint inhibitor is administered at an amount of about 3 mg/kg. In embodiments, the checkpoint inhibitor is administered at an amount of about 5 mg/kg.

In embodiments, the checkpoint inhibitor is administered at an amount of about 0.1 to about 5 mg. In embodiments, the checkpoint inhibitor is administered at an amount of 0.1 to 5 mg. In embodiments, the checkpoint inhibitor is administered at an amount of 0.1, 1, 3, or 5 mg. In embodiments, the checkpoint inhibitor is administered at an amount of 0.1 mg. In embodiments, the checkpoint inhibitor is administered at an amount of 1 mg. In embodiments, the checkpoint inhibitor is administered at an amount of 3 mg. In embodiments, the checkpoint inhibitor is administered at an amount of 5 mg. In embodiments, the checkpoint inhibitor is administered at an amount of about 0.1 mg. In embodiments, the checkpoint inhibitor is administered at an amount of about 1 mg. In embodiments, the checkpoint inhibitor is administered at an amount of about 3 mg. In embodiments, the checkpoint inhibitor is administered at an amount of about 5 mg.

In embodiments, the method includes independently administering the anti-STAT3-TLR9-binding conjugate and the checkpoint inhibitor peritumorally, intraperitoneally, or intravenously. In embodiments, the anti-STAT3-TLR9-binding conjugate is administered peritumorally, intraperitoneally, or intravenously. In embodiments, the checkpoint inhibitor is administered peritumorally, intraperitoneally, or intravenously. In embodiments, the anti-STAT3-TLR9-binding conjugate is administered peritumorally. In embodiments, the anti-STAT3-TLR9-binding conjugate is administered intraperitoneally. In embodiments, the anti-STAT3-TLR9-binding conjugate is administered intravenously. In embodiments, the checkpoint inhibitor is administered intraperitoneally. In embodiments, the checkpoint inhibitor is administered peritumorally. In embodiments, the checkpoint inhibitor is administered intravenously.

In embodiments, the method includes independently administering the anti-STAT3-TLR9-binding conjugate and the PARP inhibitor peritumorally, intraperitoneally, or intravenously. In embodiments, the PARP inhibitor is administered peritumorally, intraperitoneally, or intravenously. In embodiments, the checkpoint inhibitor is administered intraperitoneally. In embodiments, the PARP inhibitor is administered peritumorally. In embodiments, the PARP inhibitor is administered intravenously.

In embodiments, the anti-STAT3-TLR9-binding conjugate and the checkpoint inhibitor are independently administered over the course of at least 40 days. In embodiments, the anti-STAT3-TLR9-binding conjugate and the checkpoint inhibitor are independently administered over the course of at least 45 days. In embodiments, the anti-STAT3-TLR9-binding conjugate and the checkpoint inhibitor are independently administered over the course of at least 50 days. In embodiments, the anti-STAT3-TLR9-binding conjugate and the checkpoint inhibitor are independently administered over the course of at least 55 days. In embodiments, the anti-STAT3-TLR9-binding conjugate and the checkpoint inhibitor are independently administered over the course of at least 60 days. In embodiments, the anti-STAT3-TLR9-binding conjugate is administered over the course of at least 40 days. In embodiments, the anti-STAT3-TLR9-binding conjugate is administered over the course of at least 45 days. In embodiments, the anti-STAT3-TLR9-binding conjugate is administered over the course of at least 50 days. In embodiments, the anti-STAT3-TLR9-binding conjugate is administered over the course of at least 55 days. In embodiments, the anti-STAT3-TLR9-binding conjugate is administered over the course of at least 60 days. In embodiments, the checkpoint inhibitor is administered over the course of at least 40 days. In embodiments, the checkpoint inhibitor is administered over the course of at least 45 days. In embodiments, the checkpoint inhibitor is administered over the course of at least 50 days. In embodiments, the checkpoint inhibitor is administered over the course of at least 55 days. In embodiments, the checkpoint inhibitor is administered over the course of at least 60 days. In embodiments, the anti-STAT3-TLR9-binding conjugate and the checkpoint inhibitor are independently administered over the course of about 56 days. In embodiments, the anti-STAT3-TLR9-binding conjugate is administered over the course of about 56 days. In embodiments, the checkpoint inhibitor is administered over the course of about 56 days.

In embodiments, the anti-STAT3-TLR9-binding conjugate and the PARP inhibitor are independently administered over the course of at least 40 days. In embodiments, the anti-STAT3-TLR9-binding conjugate and the PARP inhibitor are independently administered over the course of at least 45 days. In embodiments, the anti-STAT3-TLR9-binding conjugate and the PARP inhibitor are independently administered over the course of at least 50 days. In embodiments, the anti-STAT3-TLR9-binding conjugate and the PARP inhibitor are independently administered over the course of at least 55 days. In embodiments, the anti-STAT3-TLR9-binding conjugate and the PARP inhibitor are independently administered over the course of at least 60 days. In embodiments, the PARP inhibitor is administered over the course of at least 40 days. In embodiments, the PARP inhibitor is administered over the course of at least 45 days. In embodiments, the PARP inhibitor is administered over the course of at least 50 days. In embodiments, the PARP inhibitor is administered over the course of at least 55 days. In embodiments, the PARP inhibitor is administered over the course of at least 60 days. In embodiments, the anti-STAT3-TLR9-binding conjugate and the PARP inhibitor are independently administered over the course of about 56 days. In embodiments, the PARP inhibitor is administered over the course of about 56 days.

In embodiments, the anti-STAT3-TLR9-binding conjugate is administered every other day. In embodiments, the anti-STAT3-TLR9-binding conjugate is administered at day 2, 4, 9, 11, 16, or 18. In embodiments, the anti-STAT3-TLR9-binding conjugate is administered at day 2, 4, 9, 11, 16, 18, 23 or 25. In embodiments, the anti-STAT3-TLR9-binding conjugate is administered at day 2. In embodiments, the anti-STAT3-TLR9-binding conjugate is administered at day 4. In embodiments, the anti-STAT3-TLR9-binding conjugate is administered at day 9. In embodiments, the anti-STAT3-TLR9-binding conjugate is administered at day 11. In embodiments, the anti-STAT3-TLR9-binding conjugate is administered at day 16. In embodiments, the anti-STAT3-TLR9-binding conjugate is administered at day 18. In embodiments, the anti-STAT3-TLR9-binding conjugate is administered at day 23. In embodiments, the anti-STAT3-TLR9-binding conjugate is administered at day 25. In embodiments, the anti-STAT3-TLR9-binding conjugate is administered at day 2, 4, 9, 11, 16, and 18. In embodiments, the anti-STAT3-TLR9-binding conjugate is administered at day 2, 4, 9, 11, 16, 18, 23 and 25.

In embodiments, the method of treating a cancer in a subject includes administering an effective amount of an anti-STAT3-Toll-like receptor 9 (TLR9)-binding conjugate and an effective amount of a checkpoint inhibitor to the subject, wherein the subject is a mammal. In embodiments, the method of treating a cancer in a subject includes administering an anti-STAT3-Toll-like receptor 9 (TLR9)-binding conjugate and a checkpoint inhibitor to the subject in a combined effective amount, wherein the subject is a mammal. In embodiments, the subject is a human. In embodiments, the method of treating a cancer in a subject includes administering an effective amount of an anti-STAT3-Toll-like receptor 9 (TLR9)-binding conjugate and an effective amount of a PARP inhibitor to the subject, wherein the subject is a mammal. In embodiments, the method of treating a cancer in a subject includes administering an anti-STAT3-Toll-like receptor 9 (TLR9)-binding conjugate and a PARP inhibitor to the subject in a combined effective amount, wherein the subject is a mammal. In embodiments, the subject is a human. In embodiments, the cancer is lymphoma, head and neck cancer, pancreatic cancer, prostate cancer, breast cancer, sarcoma, glioma, ovarian cancer, gastric cancer, cervical cancer, melanoma, or colon cancer. In embodiments, the cancer is lymphoma. In embodiments, the cancer is head and neck cancer. In embodiments, the cancer is pancreatic cancer. In embodiments, the cancer is prostate cancer. In embodiments, the cancer is breast cancer. In embodiments, the cancer is sarcoma. In embodiments, the cancer is glioma. In embodiments, the cancer is ovarian cancer. In embodiments, the cancer is gastric cancer. In embodiments, the cancer is cervical cancer. In embodiments, the cancer is melanoma. In embodiments, the cancer is colon cancer. In embodiments, the cancer is Non-Hodgkin lymphoma. In embodiments, the cancer is T cell lymphoma or B cell lymphoma. In embodiments, the cancer is T cell lymphoma. In embodiments, the cancer is B cell lymphoma.

In an aspect a method of treating a cancer in a subject in need thereof is provided. The method includes administering to the subject: (i) an anti-STAT3-TLR9 binding conjugate including a CpG moiety bound to an anti-STAT3 siRNA through a covalent linker; and (ii) an anti-CTLA4 antibody, in a combined effective amount. Thus, the methods provided herein including embodiments thereof include administering an anti-STAT3-TLR9 binding conjugate and an anti-CTLA4 antibody in a combined effective amount. In embodiments, the anti-STAT3-TLR9-binding conjugate and the anti-CTLA4 antibody are administered simultaneously. In embodiments, the anti-STAT3-TLR9-binding conjugate and the anti-CTLA4 antibody are administered sequentially. In embodiments, the anti-STAT3-TLR9-binding conjugate is administered at a first time point and the anti-CTLA4 antibody is administered at a second time point, wherein the first time point precedes the second time point. In embodiments, the anti-CTLA4 antibody is administered at a first time point and the anti-STAT3-TLR9-binding conjugate is administered at a second time point, wherein the first time point precedes the second time point. In embodiments, the anti-STAT3-TLR9-binding conjugate and the anti-CTLA4 antibody are admixed prior to administration. In embodiments, the anti-STAT3-TLR9-binding conjugate and the anti-CTLA4 antibody are independently administered peritumorally, intraperitoneally, or intravenously. In embodiments, the anti-STAT3-TLR9-binding conjugate and the anti-CTLA4 antibody are administered peritumorally. In embodiments, the anti-STAT3-TLR9-binding conjugate and the anti-CTLA4 antibody are administered intraperitoneally. In embodiments, the anti-STAT3-TLR9-binding conjugate and the anti-CTLA4 antibody are administered intravenously. In embodiments, the anti-CTLA4 antibody is administered intraperitoneally. In embodiments, the anti-STAT3-TLR9-binding conjugate and the anti-CTLA4 antibody are independently administered over the course of at least 40 days. In embodiments, the anti-STAT3-TLR9-binding conjugate and the anti-CTLA4 antibody are independently administered over the course of about 56 days. In embodiments, the cancer is T cell lymphoma or B cell lymphoma.

### Pharmaceutical Compositions

In an aspect, a pharmaceutical composition including an anti-STAT3-Toll-like receptor 9 (TLR9)-binding conjugate, a checkpoint inhibitor and a pharmaceutically acceptable excipient is provided. The anti-STAT3-TLR9-binding conjugate and the checkpoint inhibitor are present in a combined synergistic amount, wherein the combined synergistic amount is effective to treat cancer in a subject in need thereof. In embodiments, the effective amount is a combined synergistic amount. Any of the anti-STAT3-TLR9 binding conjugates and the checkpoint inhibitors provided herein, including embodiments thereof, may be used for the pharmaceutical compositions provided herein. In embodiments, the anti-STAT3-TLR9 binding conjugate includes a CpG moiety bound to an anti-STAT3 siRNA through a covalent linker. In embodiments, the checkpoint inhibitor is an antibody or a small molecule. In embodiments, the checkpoint inhibitor is an antibody. In embodiments, the checkpoint inhibitor is a small molecule. In embodiments, the checkpoint inhibitor is an anti-CTLA4 antibody, an anti-PD-1 antibody or an anti-PD-L1 antibody. In embodiments, the checkpoint inhibitor is an anti-CTLA4 antibody. In embodiments, the checkpoint inhibitor is an anti-PD-1 antibody. In embodiments, the checkpoint inhibitor is an anti-PD-L1 antibody.

In another aspect, a pharmaceutical composition including an anti-STAT3-Toll-like receptor 9 (TLR9)-binding conjugate, a PARP inhibitor and a pharmaceutically acceptable excipient is provided. The anti-STAT3-TLR9-binding conjugate and the PARP inhibitor are present in a combined synergistic amount, wherein the combined synergistic amount is effective to treat cancer in a subject in need thereof. In embodiments, the effective amount is a combined synergistic amount. Any of the anti-STAT3-TLR9 binding conjugates and the PARP inhibitors provided herein, including embodiments thereof, may be used for the pharmaceutical compositions provided herein. In embodiments, the PARP inhibitor is an antibody or a small molecule. In embodiments, the PARP inhibitor is an antibody. In embodiments, the PARP inhibitor is a small molecule.

In another aspect, a pharmaceutical composition is provided. The pharmaceutical composition includes an anti-STAT3-TLR9 binding conjugate including a CpG moiety bound to an anti-STAT3 siRNA through a covalent linker, an anti-CTLA4 antibody and a pharmaceutically acceptable excipient, wherein the anti-STAT3-TLR9-binding conjugate and the anti-CTLA4 antibody are present in a combined synergistic amount, wherein the combined synergistic amount is effective to treat cancer in a subject in need thereof.

In another aspect, a pharmaceutical composition is provided. The pharmaceutical composition includes an anti-STAT3-TLR9 binding conjugate including a CpG moiety bound to an anti-STAT3 siRNA through a covalent linker, an anti-CTLA4 antibody and a pharmaceutically acceptable excipient, wherein the anti-STAT3-TLR9-binding conjugate and the anti-CTLA4 antibody are present in a combined effective amount, wherein the combined effective amount is effective to treat cancer in a subject in need thereof.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: CpG Stat3 siRNA as a Powerful Adjuvant for CTLA4 Antibody Therapy for Cancer

The disclosure provides a novel method for cancer immunotherapy: combining CpG-STAT3 siRNA with an anti-CTLA4 antibody for treating cancer. Although PD-1 and CTLA4 antibodies are considered among the most effective anti-cancer drugs, they only provide life-saving benefits to a fraction of the treated patients. Identifying new drugs/approaches that can boost the antitumor efficacies of these checkpoint blockades are critical to increase the utility of CTLA4 and PD-1 antibodies for effectively treating cancers. STAT3 operates in both cancer cells and tumor-associated immune cells to promoter cancer progression. As a transcription factor, it is a critical but difficult target for pharmacological inhibition. The STAT3 inhibitor, CpG-STAT3 siRNA, is provided herein is highly specific and able to target malignant B cells (B cell lymphoma), myeloid cells (acute myeloid leukemia and multiple myeloma) and tumor-associated immune cells including B cells, myeloid cells and DCs.

However, whether targeting STAT3 with CpG-STAT3 siRNA can boost the antitumor efficacies of CTLA-4 antibodies has not been determined. FIGS. 1-3 show evidence that administration of a combination of CpG-STAT3 siRNA with anti-CTLA4 antibody leads to significant antitumor effects and CD8 T cell tumor cell killing activities (FIG. 1).

### Example 2: Materials and Methods

### Cell Culture

Mouse A20 B cell lymphoma cells and human H9 T cell lymphoma cells were purchased from ATCC. A20 cells were culture using RPMI 1640 medium containing 10% FBS and 0.05 mM 2-mercaptoethanol. For H9 cell culture, the medium for this cell line is RPMI 1640 medium with 10% FBS. All cells were cultured in a humidified incubator 37° C. and 5% CO₂.

### In Vivo Experiments

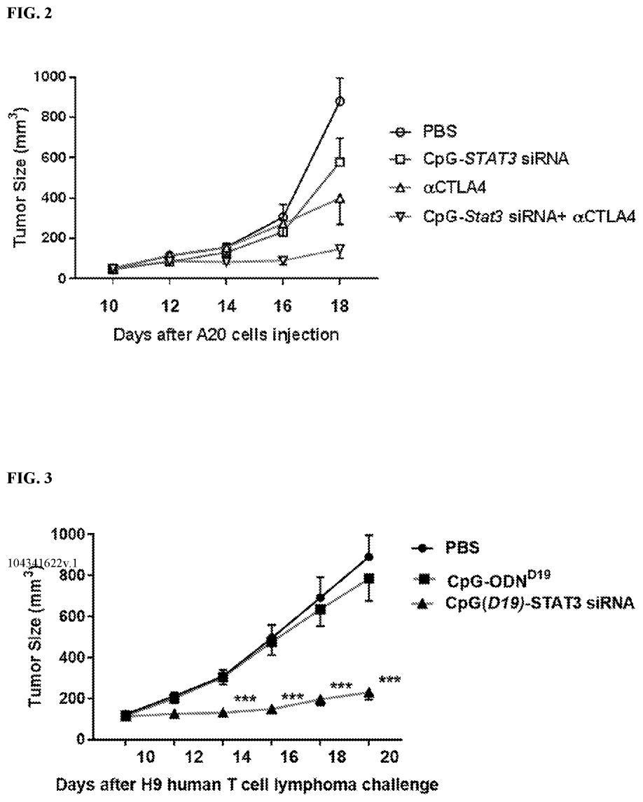

Mouse care and experimental procedures were performed under pathogen-free conditions in accordance with established institutional guidance and approved IACUC protocols from Research Animal Care Committees of the City of Hope. BALB/c mice were purchased from Jackson Laboratory. NSG (NOD/scid-IL-2Rγ_{c}^{null}) mice obtained from animal resource core facility at the City of Hope. For subcutaneous (s.c.) A20 tumor challenge, 5×10⁵ A20 cells were injected into 7- to 8-week-old BALB/c mice. After tumors reached average size of approximately 7-8 mm, mice received every other day peritumoral injections of CpG-Stat3 siRNA (15 ug/mouse) or intraperitoneally (i.p.) injection of anti-mouse CTLA4 antibody (BioxCell, 100 ug/mouse). For H9 tumor challenge, 5×10⁶ H9 cells were transplanted subcutaneously (s.c.) on the right flank of NSG mice. After tumors reached an average size of 100-150 mm³, NSG mice were locally (intratumoral injection) treated with 782.5 pmol of CpG-ODN^{D19} or CpG (D19)-STAT3 siRNA every other day. Control animals were treated with the equal volume of HBSS. Tumor growth was monitored every other day with caliper measurements (FIGS. 2 and 3).

### Intracellular Staining and Flow Cytometry

Single-cell suspensions from tumors were stimulated for 4 h with PMA (5 ng/ml, Sigma) and ionomycin (500 ng/ml, Sigma) in the presence of protein transport inhibitor (monensin 1000×, Biolegend). Cells were blocked with CD16/CD32 and incubated for 15 min on ice with FITC-, PE-, or APC-Cy7-conjugated antibodies (CD4 and CD8) purchased from Biolegend. After cells were fixed and permeabilized using the BD Cttofix/Cytoperm buffer, cells were stained with fluorescent antibodies (IFNγ and granzyme B) purchased from Biolegend. Aqua Live/DEAD used for cell viability was purchased from Invitrogen. Cells were washed twice before analysis on the BD Fortessa flow cytometer.

### Statistical Analysis

Statistical comparisons between groups were performed using the unpaired Student's t test to calculate two-tailed p-value. Statistical significance values were set as *p<0.05. **p<0.01. ***p<0.001. Data are presented as mean±standard error of the mean (SEM).

### Embodiments

Embodiment 1. A method of treating a cancer in a subject in need thereof, said method comprising administering to said subject an effective amount of an anti-STAT3-Toll-like receptor 9 (TLR9)-binding conjugate and an effective amount of a checkpoint inhibitor.

Embodiment 2. The method of embodiment 1, wherein said anti-STAT3-TLR9 binding conjugate comprises a CpG moiety bound to an anti-STAT3 siRNA through a covalent linker.

Embodiment 3. The method of any one of embodiments 1-2, wherein said checkpoint inhibitor is an antibody or a small molecule.

Embodiment 4. The method of any one of embodiments 1-3, wherein said checkpoint inhibitor is an antibody.

Embodiment 5. The method of any one of embodiments 1-4, wherein said checkpoint inhibitor is an anti-CTLA4 antibody, an anti-PD-1 antibody or an anti-PD-L1 antibody.

Embodiment 6. The method of any one of embodiments 1-5, wherein said checkpoint inhibitor is an anti-CTLA4 antibody.

Embodiment 7. The method of any one of embodiments 1-6, wherein said anti-STAT3-TLR9-binding conjugate and said checkpoint inhibitor are administered in a combined synergistic amount.

Embodiment 8. The method of any one of embodiments 1-7, wherein said anti-STAT3-TLR9-binding conjugate and said checkpoint inhibitor are administered simultaneously.

Embodiment 9. The method of any one of embodiments 1-7, wherein said anti-STAT3-TLR9-binding conjugate and said checkpoint inhibitor are administered sequentially.

Embodiment 10. The method of embodiment 9, wherein said anti-STAT3-TLR9-binding conjugate is administered at a first time point and said checkpoint inhibitor is administered at a second time point, wherein said first time point precedes said second time point.

Embodiment 11. The method of embodiment 9, wherein said checkpoint inhibitor is administered at a first time point and said anti-STAT3-TLR9-binding conjugate is administered at a second time point, wherein said first time point precedes said second time point.

Embodiment 12. The method of any one of embodiments 1-8, wherein said anti-STAT3-TLR9-binding conjugate and said checkpoint inhibitor are admixed prior to administration.

Embodiment 13. The method of any one of embodiments 1-12, wherein said anti-STAT3-TLR9-binding conjugate is administered at an amount of about 10 mg to about 40 mg.

Embodiment 14. The method of any one of embodiments 1-13, wherein said anti-STAT3-TLR9-binding conjugate is administered at an amount of 15 mg.

Embodiment 15. The method of any one of embodiments 1-13, wherein said anti-STAT3-TLR9-binding conjugate is administered at an amount of 30 mg.

Embodiment 16. The method of any one of embodiments 1-15, wherein said anti-STAT3-TLR9-binding conjugate and said checkpoint inhibitor are independently administered peritumorally, intraperitoneally, or intravenously.

Embodiment 17. The method of any one of embodiments 1-16, wherein said anti-STAT3-TLR9-binding conjugate is administered peritumorally.

Embodiment 18. The method of any one of embodiments 1-17, wherein said checkpoint inhibitor is administered intraperitoneally.

Embodiment 19. The method of any one of embodiments 1-18, wherein said anti-STAT3-TLR9-binding conjugate and said checkpoint inhibitor are independently administered over the course of at least 40 days.

Embodiment 20. The method of any one of embodiments 1-19, wherein said anti-STAT3-TLR9-binding conjugate and said checkpoint inhibitor are independently administered over the course of about 56 days.

Embodiment 21. The method of embodiment 20, wherein said anti-STAT3-TLR9-binding conjugate is administered every other day.

Embodiment 22. The method of embodiment 20, wherein said anti-STAT3-TLR9-binding conjugate is administered at day 2, 4, 9, 11, 16, or 18.

Embodiment 23. The method of embodiment 20 or 22, wherein said anti-STAT3-TLR9-binding conjugate is administered at day 2, 4, 9, 11, 16, and 18.

Embodiment 24. The method of embodiment 20, wherein said anti-STAT3-TLR9-binding conjugate is administered at day 2, 4, 9, 11, 16, 18, 23 or 25.

Embodiment 25. The method of embodiment 20 or 24, wherein said anti-STAT3-TLR9-binding conjugate is administered at day 2, 4, 9, 11, 16, 18, 23 and 25.

Embodiment 26. The method of any one of embodiments 1-25, wherein said subject is a mammal.

Embodiment 27. The method of any one of embodiments 1-26, wherein said subject is a human.

Embodiment 28. The method of any one of embodiments 1-27, wherein said cancer is lymphoma.

Embodiment 29. The method of any one of embodiments 1-28, wherein said cancer is Non-Hodgkin lymphoma.

Embodiment 30. A pharmaceutical composition comprising an anti-STAT3-Toll-like receptor 9 (TLR9)-binding conjugate, a checkpoint inhibitor and a pharmaceutically acceptable excipient, wherein said anti-STAT3-TLR9-binding conjugate and said checkpoint inhibitor are present in a combined effective amount, wherein said combined effective amount is effective to treat cancer in a subject in need thereof.

Embodiment 31. The pharmaceutical composition of embodiment 30, wherein said effective amount is a combined synergistic amount.

Embodiment 32. The pharmaceutical composition of embodiment 30 or 31, wherein said anti-STAT3-TLR9 binding conjugate comprises a CpG moiety bound to an anti-STAT3 siRNA through a covalent linker.

Embodiment 33. The pharmaceutical composition of any one of embodiments 30-32, wherein said checkpoint inhibitor is an antibody or a small molecule.

Embodiment 34. The pharmaceutical composition of any one of embodiments 30-33, wherein said checkpoint inhibitor is an antibody.

Embodiment 35. The pharmaceutical composition of any one of embodiments 30-34, wherein said checkpoint inhibitor is an anti-CTLA4 antibody, an anti-PD-1 antibody or an anti-PD-L1 antibody.

Embodiment 36. The pharmaceutical composition of any one of embodiments 30-35, wherein said checkpoint inhibitor is an anti-CTLA4 antibody.

What is claimed is:

1. A method of treating a cancer in a subject in need thereof, said method comprising administering to said subject:
(i) an anti-STAT3-TLR9 binding conjugate comprising a CpG moiety bound to an anti-STAT3 siRNA through a covalent linker; and
(ii) an anti-CTLA4 antibody, in a combined effective amount.

2. The method of claim 1, wherein said combined effective amount is a combined synergistic amount.

3. The method of claim 1, wherein said anti-STAT3-TLR9-binding conjugate and said anti-CTLA4 antibody are administered simultaneously.

4. The method of claim 1, wherein said anti-STAT3-TLR9-binding conjugate and said anti-CTLA4 antibody are administered sequentially.

5. The method of claim 4, wherein said anti-STAT3-TLR9-binding conjugate is administered at a first time point and said anti-CTLA4 antibody is administered at a second time point, wherein said first time point precedes said second time point.

6. The method of claim 4, wherein said anti-CTLA4 antibody is administered at a first time point and said anti-STAT3-TLR9-binding conjugate is administered at a second time point, wherein said first time point precedes said second time point.

7. The method of claim 1, wherein said anti-STAT3-TLR9-binding conjugate and said anti-CTLA4 antibody are admixed prior to administration.

8. The method of claim 1, wherein said anti-STAT3-TLR9-binding conjugate is administered at an amount of about 10 mg to about 40 mg.

9. The method of claim 1, wherein said anti-STAT3-TLR9-binding conjugate is administered at an amount of 15 mg.

10. The method of claim 1, wherein said anti-STAT3-TLR9-binding conjugate is administered at an amount of 30 mg.

11. The method of claim 1, wherein said anti-STAT3-TLR9-binding conjugate and said anti-CTLA4 antibody are independently administered peritumorally, intraperitoneally, or intravenously.

12. The method of claim 1, wherein said anti-STAT3-TLR9-binding conjugate is administered peritumorally.

13. The method of claim 1, wherein said anti-CTLA4 antibody is administered intraperitoneally.

14. The method of claim 1, wherein said anti-STAT3-TLR9-binding conjugate and said anti-CTLA4 antibody are independently administered over the course of at least 40 days.

15. The method of claim 1, wherein said anti-STAT3-TLR9-binding conjugate and said anti-CTLA4 antibody are independently administered over the course of about 56 days.

16. The method of claim 15, wherein said anti-STAT3-TLR9-binding conjugate is administered every other day.

17. The method of claim 15, wherein said anti-STAT3-TLR9-binding conjugate is administered at day 2, 4, 9, 11, 16, or 18.

18. The method of claim 15, wherein said anti-STAT3-TLR9-binding conjugate is administered at day 2, 4, 9, 11, 16, and 18.

19. The method of claim 15, wherein said anti-STAT3-TLR9-binding conjugate is administered at day 2, 4, 9, 11, 16, 18, 23 or 25.

20. The method of claim 15, wherein said anti-STAT3-TLR9-binding conjugate is administered at day 2, 4, 9, 11, 16, 18, 23 and 25.

21. The method of claim 1, wherein said subject is a mammal.

22. The method of claim 1, wherein said subject is a human.

23. The method of claim 1, wherein said cancer is lymphoma.

24. The method of claim 1, wherein said cancer is T cell lymphoma or B cell lymphoma.

25. The method of claim 1, wherein said cancer is Non-Hodgkin lymphoma.

26. A pharmaceutical composition comprising an anti-STAT3-TLR9 binding conjugate comprising a CpG moiety bound to an anti-STAT3 siRNA through a covalent linker, an anti-CTLA4 antibody and a pharmaceutically acceptable excipient, wherein said anti-STAT3-TLR9-binding conjugate and said anti-CTLA4 antibody are present in a combined effective amount, wherein said combined effective amount is effective to treat cancer in a subject in need thereof.

27. The pharmaceutical composition of claim 26, wherein said combined effective amount is a combined synergistic amount.


SEQUENCE LISTING (19 sequences)

SEQ ID NO: 1 - DNA, 20 bp, Artificial Sequence
  Synthetic polynucleotide
  misc_feature (1)..(3): Residue modified with phosphorothioate
  misc_feature (16)..(20): Residue modified with phosphorothioate
ggtgcatcgatgcagggggg

SEQ ID NO: 2 - RNA, 27 nt, Artificial Sequence
  Synthetic polynucleotide
ucagucguaucuuucugcagcuuccgu

SEQ ID NO: 3 - RNA, 25 nt, Artificial Sequence
  Synthetic polynucleotide
ggaagcugcagaaagauacgacuga

SEQ ID NO: 4 - DNA, 20 bp, Artificial Sequence
  Synthetic polynucleotide
  misc_feature (1)..(3): Residue modified with phosphorothioate
  misc_feature (16)..(20): Residue modified with phosphorothioate
ggtgcatcgatgcagggggg

SEQ ID NO: 5 - DNA, 20 bp, Artificial Sequence
  Synthetic polynucleotide
  misc_feature (1)..(3): Residue modified with phosphorothioate
  misc_feature (16)..(20): Residue modified with phosphorothioate
ggtgcatgcatgcagggggg

SEQ ID NO: 6 - DNA, 20 bp, Artificial Sequence
  Synthetic polynucleotide
  misc_feature (1)..(20): Residue modified with phosphorothioate
ggtgcatcgatgcagggggg

SEQ ID NO: 7 - DNA, 20 bp, Artificial Sequence
  Synthetic polynucleotide
  misc_feature (1)..(20): Residue modified with phosphorothioate
tccatgacgttcctgatgct

SEQ ID NO: 8 - DNA, 20 bp, Artificial Sequence
  Synthetic polynucleotide
  misc_feature (1)..(2): Residue modified with phosphorothioate
  misc_feature (3)..(3): Residue optionally modified with phosphorothioate
  misc_feature (16)..(20): Residue modified with phosphorothioate
ggggtcaacgttgagggggg

SEQ ID NO: 9 - DNA, 20 bp, Artificial Sequence
  Synthetic polynucleotide
  misc_feature (1)..(2): Residue modified with phosphorothioate
  misc_feature (3)..(3): Residue optionally modified with phosphorothioate
  misc_feature (16)..(20): Residue modified with phosphorothioate
gggggacgatcgtcgggggg

SEQ ID NO: 10 - DNA, 20 bp, Artificial Sequence
  Synthetic polynucleotide
  misc_feature (1)..(2): Residue modified with phosphorothioate
  misc_feature (3)..(3): Residue optionally modified with phosphorothioate
  misc_feature (16)..(20): Residue modified with phosphorothioate
ggtgcatcgatgcagggggg

SEQ ID NO: 11 - DNA, 21 bp, Artificial Sequence
  Synthetic polynucleotide
  misc_feature (1)..(3): Residue modified with phosphorothioate
  misc_feature (4)..(4): Residue optionally modified with phosphorothioate
  misc_feature (16)..(21): Residue modified with phosphorothioate
ggggacgacgtcgtggggggg

SEQ ID NO: 12 - DNA, 20 bp, Artificial Sequence
  Synthetic polynucleotide
  misc_feature (1)..(20): Phosphorothioate linkage between residues 1-20.
tccatgacgttcctgatgct

SEQ ID NO: 13 - DNA, 20 bp, Artificial Sequence
  Synthetic polynucleotide
  misc_feature (1)..(20): Phosphorothioate linkage between residues 1-20.
tccatgacgttcctgacgtt

SEQ ID NO: 14 - DNA, 24 bp, Artificial Sequence
  Synthetic polynucleotide
  misc_feature (1)..(24): Phosphorothioate linkage between residues 1-24.
tcgtcgttttgtcgttttgtcgtt

SEQ ID NO: 15 - DNA, 22 bp, Artificial Sequence
  Synthetic polynucleotide
  misc_feature (1)..(22): Phosphorothioate linkage between residues 1-22.
tcgtcgttgtcgttttgtcgtt

SEQ ID NO: 16 - DNA, 22 bp, Artificial Sequence
  Synthetic polynucleotide
  misc_feature (1)..(22): Phosphorothioate linkage between residues 1-22.
tcgtcgttttcggcgcgcgccg

SEQ ID NO: 17 - DNA, 25 bp, Artificial Sequence
  Synthetic polynucleotide
  misc_feature (1)..(25): Phosphorothioate linkage between residues 1-25.
tcgtcgtcgttcgaacgacgttgat

SEQ ID NO: 18 - RNA, 25 nt, Artificial Sequence
  Synthetic polynucleotide
  misc_feature (3)..(4): Residues 3-4 are 2′OMe, 2′F, LNA or otherwise modified residues
  misc_feature (6)..(7): Residues 6-7 are 2′OMe, 2′F, LNA or otherwise modified residues
  misc_feature (9)..(10): Residues 9-10 are 2′OMe, 2′F, LNA or otherwise modified residues
  misc_feature (12)..(14): Residues 12-14 are 2′OMe, 2′F, LNA or otherwise modified residues
  misc_feature (16)..(19): Residues 16-19 are 2′OMe, 2′F, LNA or otherwise modified residues
  misc_feature (23)..(23): Residue 23 is a 2′OMe, 2′F, LNA or otherwise modified residue
ggaagcugcagaaagauacgacuga

SEQ ID NO: 19 - DNA, 6 bp, Artificial Sequence
  Synthetic polynucleotide
rycgyr